(19) United States
(12) Patent Application Publication
(10) Pub. No.: US 2012/0064044 A1
(43) Pub. Date: Mar. 15, 2012

(54) METHODS OF TREATING EDEMA RELATED TO ISCHEMIA-REPERFUSION

(76) Inventor: Thomas Michael Egan, Chapel Hill, NC (US)

(21) Appl. No.: 13/263,435

(22) PCT Filed: Apr. 9, 2010
(86) PCT No.: PCT/US10/30556

§ 371 Date: Nov. 28, 2011

Related U.S. Application Data
(60) Provisional application No. 61/168,089, filed on Apr. 9, 2009.

(51) Int. Cl. A61K 31/7028; C07H 15/04; A61K 35/12; A61K 35/34; A61K 35/42; A61P 7/10; A61K 35/23; A61K 35/407; A61K 35/37; A61K 35/39; A61K 35/36; C07H 15/14; A61K 35/30

(52) U.S. Cl. 424/93.7; 514/24; 514/25; 536/17.1

(57) ABSTRACT

Methods are described for preventing or reducing edema related to ischemia-reperfusion by treating the organ or tissue being transplanted with an aminoalkyl glucosaminide phosphate.

### RELATED APPLICATIONS

This application claims the benefit of U.S. Patent Application Ser. No. 61/168,089, filed Apr. 9, 2009, the disclosure of which is incorporated herein by reference in its entirety.

### TECHNICAL FIELD

The presently disclosed subject matter relates to methods and compositions for preventing or reducing edema related to ischemia-reperfusion.

### Abbreviations

° C.=degrees Celsius
AGP=aminoalkyl glucosaminide phosphate
AMs=alveolar macrophages
ARDS=Adult Respiratory Distress Syndrome
BAL=bronchoalveolar lavage
BMT=bone marrow transplant
β-gal=β-galactosidase
CO₂=carbon dioxide
dpi=dots per inch
EBD=Evans blue dye
EDTA=ethylenediamine tetraacetic acid
EGTA=ethylene glycol tetraacetic acid
FBS=fetal bovine serum
FiO₂=fraction of inspired oxygen
Fluc=firefly luciferase
g=relative centrifuge force
Gy=gray
HCl=hydrochloric acid
HEPES=N-2-hydroxyethylpiperazine-N′-2-ethanesulfonic acid
HMVECs=human pulmonary microvascular endothelial cells
hr=hours
ICAM-1=intercellular adhesion molecule-1
IRI=ischemia-reperfusion injury
LDH=lactate dehydrogenase
LPS=lipopolysaccharide
MAPKs=mitogen activated protein kinases
μg=microgram
μL=microliter
μm=micron
μM=micromolar
mg=milligram
min=minutes
mL=milliliter
NF-κB=nuclear factor-kappa B
NHBD non-heart-beating donor
nm=nanometer
O₂=oxygen
PAMP=pathogen-associated molecular patterns
PBS=phosphate buffered saline
PEEP=positive end-expiratory pressure
PGN=peptidoglycan
PMSF=phenylmethanesulphonylfluoride
RPMI=Roswell Park Memorial Institute
SDS-PAGE=sodium dodecyl sulfate polyacrylamide gel electrophoresis
SIRS=Systemic Inflammatory Response Syndrome
TBS=tris-buffered saline
TLR2=Toll-like Receptor 2
TLR4 Toll-like Receptor 4
TPCK=L-1-tosylamido-2-phenylethyl chloromethyl ketone
Tris=tris(hydroxymethyl)amino methane
W/D=wet to dry weight ratio

### BACKGROUND

Acute lung injury is a feature of sepsis, systemic inflammatory response, and adult respiratory distress syndrome. Non-cardiogenic pulmonary edema and impaired gas exchange are consequences of acute lung injury, irrespective of etiology. The mechanisms causing pulmonary edema due to acute lung injury are not well understood. lschemia-reperfusion injury (IRO, a form of acute lung injury occurring immediately following lung transplantation, is a frequent complication causing morbidity and mortality. See King et al., Ann. Thorac. Surg., 69, 1681-1685 (2000).

Reperfusion following an interval of ischemia results in an inflammatory response involving components of the innate immune system, including the complement and coagulation cascades. Both parenchymal and myeloid cells elaborate free radicals, nitric oxide, and pro- and anti-inflammatory cytokines. See de Perrot et al., Am. J. Respir. Crit. Care Med., 167(4), 490-511 (2003); de Groot and Rauen, Transplant Proc., 39(2), 481-484 (2007); and Mollen et al., Shock, 26(5), 430-437 (2006).

A greater understanding of lung IRI is likely relevant to many types of acute lung injury, and can be of benefit to substantial numbers of patients, in addition to lung transplant recipients. In particular, such knowledge could be of benefit to patients with ischemia-reperfusion related edema in organs other than the lung.

### SUMMARY

In some embodiments, the presently disclosed subject matter provides a method of preventing or reducing edema in a tissue, the method comprising contacting the tissue with an effective amount of a compound of Formula (I):

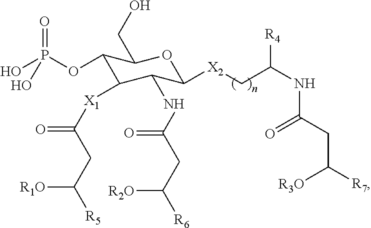

wherein:

n is an integer from 1 to 6;

X₁ is O or S;

X₂ is O or S;

R₁, R₂, and R₃ are independently C₂-C₁₆ acyl, wherein at least one of R₁, R₂, and R₃ is C₂-C₇ acyl;

R₄ is selected from the group consisting of H, hydroxylalkyl, —C(═O)NH₂, and —(CH₂)ₘC(═O)OH, wherein m is an integer from 0 to 2; and

R₅, R₆, and R₇ are independently C₁₀-C₁₂ alkyl, or a pharmaceutically acceptable salt thereof.

In some embodiments, n is 1. In some embodiments, X₁ and X₂ are each O. In some embodiments, R₄ is —C(═O)OH. In some embodiments, R₁, R₂, and R₃ are each C₂-C₇ acyl.

In some embodiments, the compound of Formula (I) is a compound wherein n is 1; X₁ is O; X₂ is O; R₁, R₂ and R₃ are each —C(═O)(CH₂)₄CH₃; R₄ is —C(═O)OH; and R₅, R₆, and R₇ are each —(CH₂)₁₀CH₃, or a pharmaceutically acceptable salt thereof.

In some embodiments, the edema is related to ischemia-reperfusion. In some embodiments, the ischemia-reperfusion is related to myocardial infarction or stroke. In some embodiments, the ischemia-reperfusion is related to cardioplegia during cardiac surgery or to ischemia-reperfusion in skeletal muscle resulting from orthopedic surgery. In some embodiments, the ischemia-reperfusion is related to organ or tissue transplant. In some embodiments, the tissue transplant is a skin, muscle, or soft tissue transplant. In some embodiments, the tissue transplant is an autologous tissue transplant.

In some embodiments, contacting the tissue with an effective amount of the compound occurs prior to ischemia, during ischemia, or after an interval of ischemia.

In some embodiments, the tissue is selected from the group consisting of heart, liver, kidney, brain, small or large bowel, pancreas, skeletal muscle, skin, soft tissue, and lung tissue. In some embodiments, the tissue is from an organ donor. In some embodiments, the tissue is lung tissue from a lung transplant donor. In some embodiments, the lung transplant donor is a human lung transplant donor. In some embodiments, the organ donor is a non-heart-beating donor.

In some embodiments, the compound is an antagonist of one or both of Toll-like receptor 2 (TLR2) and Toll-like receptor 4 (TLR4). In some embodiments, the compound is an antagonist of both TLR2 and TLR4.

In some embodiments, the presently disclosed subject matter provides a method of preventing or reducing edema in a subject in need of treatment thereof, the method comprising administering to the subject, an effective amount of a compound of Formula (I) or a pharmaceutically acceptable salt thereof.

In some embodiments, the presently disclosed subject matter provides a method of preventing or reducing edema related to ischemia-reperfusion in a subject of an organ or tissue transplant, the method comprising: providing an organ or tissue for transplant; contacting the organ or tissue with a compound of Formula (I) or a pharmaceutically acceptable salt thereof; and transplanting the treated organ or tissue into a subject in need of said transplant, wherein edema related to ischemia-reperfusion in the subject is prevented or reduced in comparison to edema related to ischemia-reperfusion in a subject of a transplant performed using an organ or tissue untreated with said compound.

In some embodiments, the organ or tissue is selected from the group consisting of a heart or heart tissue, a liver or liver tissue, a kidney or kidney tissue, a pancreas or pancreatic tissue, small or large bowel tissue, skeletal muscle tissue, soft tissue, a lung or lung tissue, and brain tissue. In some embodiments, the organ or tissue is a lung or lung tissue. In some embodiments, the organ or tissue is from a non-heart-beating organ donor.

In some embodiments, the contacting is performed via one of the airway of a lung tissue donor, the pulmonary vein, and the pulmonary artery of an ex vivo perfusion circuit.

In some embodiments, the tissue is one of skin tissue, skeletal muscle tissue, or soft tissue. In some embodiments, the tissue transplant is an autologous tissue transplant.

In some embodiments, the compound is an antagonist of one or both of TLR2 and TLR4. In some embodiments, the compound is an antagonist of both TLR2 and TLR4.

In some embodiments, the compound of Formula (I) is a compound wherein n is 1. In some embodiments X₁ and X₂ are each O. In some embodiments, R₄ is —C(═O)OH. In some embodiments, R₁, R₂, and R₃ are each C₂-C₇ acyl. In some embodiments, the compound of Formula (I) is a compound wherein n is 1; X₁ is O; X₂ is O; R₁, R₂ and R₃ are each —C(═O)(CH₂)₄CH₃; R₄ is —C(═O)OH; and R₅, R₆, and R₇ are each —(CH₂)₁₀CH₃, or a pharmaceutically acceptable salt thereof.

In some embodiments, the presently disclosed subject matter provides a preservation solution for treating an ex vivo organ or organ tissue comprising a compound of Formula (I) or a pharmaceutically acceptable salt thereof. In some embodiments, the ex vivo organ or tissue is a lung or a portion thereof.

It is an object of the presently disclosed subject matter to provide methods and compositions for preventing or reducing edema, such as edema related to ischemia-reperfusion.

An object of the presently disclosed subject matter having been stated hereinabove, and which is achieved in whole or in part by the presently disclosed subject matter, other objects will become evident as the description proceeds when taken in connection with the accompanying examples and drawings as best described hereinbelow.

### BRIEF DESCRIPTION OF THE DRAWINGS

FIG. 1A is a bar graph of edema (as measured by wet to dry weight ratio (W/D)) in reperfused lungs from Toll-like receptor 4 (TLR4)-sufficient (OuJ) and TLR4-deficient (HeJ) mice at 0 min, 15 min, 30 min, 1 hr, and 3 hr following reperfusion. W/D data is shown for both the left lung (LL OuJ, open bars) and right lung (RL OuJ, shaded bars) of OuJ mice and for the left lung (LL HeJ, open bars with dark dots) and right lung (RL HeJ, shaded bars with white squares) of HeJ mice. *=p<0.05, †=p<0.01 compared to controls. n=6/group.

FIG. 1B is a series of photographs of inflation fixed (25 cm H₂O) left lungs from Toll-like receptor 4 (TLR4)-sufficient (OuJ) and TLR4-deficient (HeJ) mice retrieved after 1 hour hilar clamping and 3 hours of reperfusion. The arrows in the upper pair of photographs show increased interstitial edema in peribronchial and perivascular spaces in lung from an OuJ mouse (upper left photo) compared to lung from an HeJ mouse (upper right photo). The arrows in the lower pair of photographs show thicker alveolar walls in lung from an OuJ mouse (lower left photo) compared to lung from an HeJ mouse (lower right photo). The upper photographs are shown under 40 times magnification (lines in the lower right of the upper photos represent 2.0 mm); the lower photographs are shown under 200 times magnification (lines in the lower right of the lower, photos represent 200 μm). The photos are representative of 4 specimens.

FIG. 1C is a series of photographs of inflation fixed left lungs from Toll-like receptor 4 (TLR4)-sufficient (OuJ) and TLR4-deficient (HeJ) mice retrieved after 1 hour hilar clamping and 1 hour reperfusion. Despite significant difference in wet to dry weight ratio (W/D) following 60 min reperfusion, there is no interstitial peribronchial/ perivascular edema in OuJ mouse lung (upper left photo) or HeJ mouse lung (upper right photo), and no alveolar wall thickening (lower photos). The photographs are representative of four specimens. The inflation fixed sections shown in the photographs appear identical to control specimens (not shown). The upper photographs are shown under 40 times magnification (lines in lower right of upper photos represent 2.0 mm); the lower photographs are shown under 200 times magnification (lines in lower right of lower photos represent 200 μm).

FIG. 1D is a bar graph of Evans blue dye accumulation (as measured by optical density (OD)/gram (gm) sample) in left (open bars) and right lungs (shaded bars) from both Toll-like receptor 4 (TLR4)-sufficient (OuJ) mice and TLR4-deficient (HeJ) mice retrieved after one hour of hilar clamping and one hour of reperfusion. Data for OuJ mice is shown in the pair of bars on the left side of the graph and data for HeJ mice is shown in the pair of bars on the right side of the graph. *=p<0.05 unpaired t test; ‡=p<0.05 paired t test.

FIG. 1E is a bar graph comparing edema (as measured by wet to dry weight ratio (W/D)) in left (open bars) and right (shaded bars) lungs from Toll-like receptor 4 (TLR4)-sufficient (OuJ) mice (n=6), TLR4-deficient (HeJ) mice, MyD88-deficient mice (n=5) and C57BL/6J mice (the background strain for the MyD88-deficient mice; n=6) retrieved after one hour of hilar clamping and one hour of reperfusion.

FIG. 1F is a bar graph of edema (as measured by wet to dry weight ratio (W/D)) in right lungs (RL TLR4−/−, stippled bars) and left lungs (LL TLR4−/−, unshaded bars) of Toll-like receptor 4 (TLR4)-deficient mice bred on the C57BL/6J mouse strain following 1 hour of ischemia and 0 or 5 minutes of reperfusion. Edema was also measured in right lungs (RL BL6, striped bars) and left lungs (LL BL6, darkly shaded bars) of the background strain C57BL/6J mice after one hour left hilar clamping followed by reperfusion for 0 or 5 minutes. *p<0.05 compared to right BL6 lungs and right and left lungs from TLR4−/− mice, (ANOVA with Tukey's post hoc). n=6/group.

FIG. 2A is a series of photographs of Western blotting gels showing activation of JNK, ERK, p38, and NF-κB in left lungs of control, Toll-like receptor 4 (TLR4)-sufficient (OuJ) and TLR4-deficient (HeJ) mice rendered ischemic for 1 hour and then reperfused for 0, 15, 30, 60, or 180 minutes. n=4 for each of HeJ and OuJ strains. Control represents protein extracted from lungs of 2 HeJ and 2 OuJ mice that had not undergone ischemia/reperfusion.

FIG. 2B is a series of bar graphs of the protein concentration data (intensity quantified by laser scanning) from the Western blots described for FIG. 2A. Data for Control, HeJ and OuJ mouse lung is represented by lightly shaded, darkly shaded, and open bars, respectively. Phospho/total mitogen activated protein kinases (MAPKs) and IκBα/β-actin were normalized by dividing each ratio by the mean ratio for controls, making each control=1.0, with variability among the different control samples represented by error bars (mean±SEM). p46 and p54 JNK, and p44 and p42 ERK have similar patterns and p values. *=p<0.05; †=p<0.01; ‡=p<0.001 compared to Controls by ANOVA with Tukey's Honest Significant Difference for multiple comparisons.

FIG. 3 is a series of photographs of lung tissue samples immunostained for the p65 component of NF-κB. The control sample (left-most photograph) is of immunostained lung tissue from a freshly sacrificed mouse.

The remaining photographs show immunostained lung tissue from the right (lower four photographs) and left (upper four photographs) lungs of Toll-like receptor 4 (TLR4)-sufficient (OuJ) and TLR4-deficient (HeJ) mice following 60 or 180 minutes of reperfusion. Lines in the bottom right of each photograph represent 100 μm.

FIG. 4A is a bar graph of NF-κB activation (as measured by firefly luciferase (fluc)/β-galactosidase (β-gal) activity) in alveolar macrophages (AMs) from chimeric mice twelve weeks following bone marrow transplant. The AMs were retrieved by bronchoalveolar lavage (BAL) and infected with Ad.NFκBLuc and Ad.CMV-LacZ, then incubated with either phosphate buffered saline (PBS; open bars) or 1 μg/mL lipopolysaccharide (LPS, darkly shaded bars). The observed firefly luciferase/β-galactosidase activity indicated complete replacement of recipient marrow from either chimeric HeJ strain (P+M−) or chimeric OuJ strain (P−M+). P=parenchymal cells, M=marrow-derived cells, +=intact TLR4 (OuJ), −=non-functional TLR4 (HeJ). AMs retrieved from non-irradiated HeJ and OuJ mice served as controls. n=4 experiments/group, p<0.0001.

FIG. 4B is a bar graph of edema (as measured by wet to dry weight ratio (W/D)) in lung tissue from left (open bars) and right (darkly shaded bars) lungs of chimeric mice 3 hours following IRI. P=parenchymal cells, M=marrow-derived cells, +=intact TLR4 (OuJ), −=non-functional TLR4 (HeJ). *=p<0.05, †=p<0.01 compared to W/D of P-left lungs (ANOVA with Tukey's Honest Significant Difference).

FIG. 4C is a bar graph of wet to dry weight ratio (W/D) in left (open bars) and right (darkly shaded bars) lung tissue from chimeric mice with restored bone marrow (P−M−, P+M+) and in intact HeJ and OuJ strains.

FIG. 5A is a bar graph of edema (as measured by wet to dry weight ratio (W/D)) following 1 hr of ischemia reperfusion injury (IRI) in right (darkly shaded bars) and left (open bars) lungs of OuJ mice treated intravenously for 30 minutes with either vehicle (saline, bars on left side of graph) or the TLR4 competitive inhibitor CRX-526 (10 μg in 200 μL of saline, bars on right side of graph) starting one hour before hilar clamping. N=5/group, *=p=0.0014 compared to right lung of the same animal by paired t test; p=0.0023 compared to left lung of mice pre-treated with CRX-526 by unpaired t test.

FIG. 5B is a bar graph showing the in vitro inhibition of NF-κB activation (based on firefly luciferase (fluc)/β-galactosidase (β-gal) activity) effected by 0.1, 1, 10, and 100 μg concentrations (i.e., Inhib 0.1, Inhib 1, Inhib 10, Inhib 100, respectively) of CRX-526. Fluc/β-gal was measured following stimulation of CRX-526-treated human pulmonary microvascular endothelial cells (HMVECs) with phosphate buffered saline (PBS) only (open bars), 10 ng/mL lipopolysaccharide (LPS) (spotted bars), 5 ng/mL LPS (striped bars), or 0.25 ng/mL tumor necrosis factor (TNF, darkly shaded bars). HMVECs that had not been treated with CRX-526 were used as a control. n=4/group, *p<0.05 compared to other values at same time point by ANOVA.

FIG. 6 is a graph of edema (based on wet to dry weight ratio (W/D)) in Toll-like receptor 2 deficient mice (TLR2−/−) after 1 hour left hilar occlusion and 15, 30 or 60 minutes of reperfusion. Edema in the right lung (TLR2−/−RL) of the TLR2−/− mice is shown in the striped bars, while edema in the left lung (TLR2−/−LL) of the TLR2−/− mice is shown in the lightly shaded bars. Edema was also measured in the right (BL6 RL, open bars) and left lungs (BL6 LL, darkly shaded bars) of the background C57BL/6J mouse strain as a control. †=p<0.01; ‡=p<0.001 vs control left lung.

FIG. 7A is graph of edema (as measured by wet to dry weight ratio (W/D)) in left lungs of C57BL/6J mice (BL6, darkly shaded bars), in left lungs of Toll-like receptor 4 deficient mice (TLR4−/−, striped bars), in left lungs of Toll-like receptor 2 deficient mice (TLR2−/−, stippled bars), and in left lungs of C57BU6J mice pre-treated with 10 μg of CRX-526 for over 30 minutes starting 1 hour prior to left hilar clamping for 1 hour (CRX-526, unshaded bars). Control (Fresh) data is from murine lung retrieved immediately after animal sacrifice without hilar clamping. As indicated on the x-axis of the graph, the other data is from lungs after 1 hour of left hilar clamping and either 0, 15, 30, 60 or 180 minutes of reperfusion. n=3-6. BL6 W/D is significantly higher than in other strains or CRX-526 treated mice. *p<0.05, †p<0.01, ‡p<0.001; TLR2−/− compared to TLR4−/− and CRX-526-treated mice §p<0.01, σp<0.05 (ANOVA with Tukey's post hoc at each time point).

FIG. 7B is a graph of edema (measured by wet to dry weight ratio (W/D)) in right lungs of C57BL/6J mice (BL6, darkly shaded bars), in right lungs of Toll-like receptor 4 deficient mice (TLR4−/−, striped bars), in right lungs of Toll-like receptor 2 deficient mice (TLR2−/−, stippled bars), and in right lungs of C57BL/6J mice pre-treated with 10 μg of CRX-526 for over 30 minutes starting 1 hour prior to left hilar clamping for 1 hour (CRX-526, unshaded bars). Control (Fresh) data is from murine lung retrieved immediately after animal sacrifice and prior to hilar clamping. As indicated on the x-axis of the graph, the other data is from lungs after 1 hour of left hilar clamping and either 0, 15, 30, 60 or 180 minutes of reperfusion. Data n=3-6.

FIG. 8 is a bar graph showing the effects of CRX-526 on NF-κB activation mediated through stimulation by Toll-like receptor 2 (TLR2) ligands. Human pulmonary microvascular endothelial cells (HMVECs) were transfected with recombinant first-generation E1, E3-deleted Ad.NF-κB-luciferase and consituitive β-galactosidase vectors so that when NF-κB is activated, the ratio of firefly luciferase (fluc)/β-galactosidase (β-gal) increases. Forty eight hours after transfection, the HMVECs were pretreated for 1 hour with various concentrations of CRX-526 (as shown on the x-axis) and exposed to TLR2 ligands Pam(3)Cys (25 μg/mL, darkly shaded bars) or lipoteichoic acid (LTA; 1 μg/mL, open bars) for 8 hours. Luciferase activity is normalized to β-galactosidase to control for infection efficiency. CRX-526 reduced NF-κB activation. *p<0.05, †p<0.01 compared to vehicle.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a compound” or “a cell” includes a plurality of such compounds or cells, and so forth.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 6 carbon atoms (i.e., a C₁₋₇ alkyl), e.g., 1, 2, 3, 4, 5, or 6 carbon atoms. “Higher alkyl” refers to an alkyl group having about 8 to about 20 carbon atoms, e.g., 8, 9, 10, 11, 12, 13, 14, 15, 16, 17, 18, 19, or 20 carbon atoms.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “alkenyl” refers to an alkyl group comprising one or more carbon-carbon double bonds.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Hydroxy” and “hydroxyl” refer to the group —OH.

The term “hydroxyalkyl” refers to a hydroxy-terminated alkyl group. In some embodiments, the hydroxyalkyl group has the structure —(CH₂)ₙOH.

The term “carboxylic acid” refers to the group —C(═O)OH. The term “carboxylate” refers to anion formed when the H of the carboxylic acid group is removed. Thus, “carboxylate” refers to the group —C(═O)O⁻. Carboxylates can form salts (i.e., carboxylate salts) with cationic groups. The terms “alkylene carboxylate” and “alkylene carboxylic acid” refer to monovalent groups formed by the attachment of a carboxylic acid or carboxylate group to one open attachment point on an alkylene group (e.g., the groups —(CH₂)ₙC(═O)OH and —(CH₂)ₙC(═O)O⁻).

As used herein, the term “acyl” refers to the group —C(═O)R, wherein R is an alkyl or aryl group as defined hereinabove. In some embodiments, the R of the acyl group is C₁-C₁₆ alkyl. In some embodiments, the alkyl group of the acyl moiety is straight chain alkyl or alkenyl. In some embodiments the R of the acyl group is C₁-C₁₆ straight chain alkyl.

The term “phosphate” refers to the group —P(═O)(OH)₂. The term “phosphate” also includes anionic species formed by the removal of one or more hydrogen atoms of the phosphate group.

The term “thiol” refers to a group having the structure —S—R, wherein R is alkyl, acyl, or aryl. The term “thiol” can also refer to a compound having the structure H—S—R, wherein R is alkyl, acyl, or aryl.

The term “amino” refers to a group having the structure —NR₁R₂, wherein R₁ and R₂ are independently selected from the group H, alkyl, acyl, and aryl.

The term “carbamoyl” refers to the group —C(═O)NH₂.

The term “monosaccharide” refers to a carbohydrate monomer unit of the formula (CH₂O)ₙ₊ₘ based upon an open chain form of a compound having the chemical structure H(CHOH)ₙC(═O)(CHOH)ₘH, wherein the sum of n+m is an integer between 2 and 8. Thus, the monomer units can include trioses, tetroses, pentoses, hexoses, heptoses, nonoses, and mixtures thereof. The monosaccharide can be in a cyclized form of the chemical structure. Thus, in some embodiments, the compound will comprise a hemiacetal or hemiketal. In some embodiments, the term “monosaccharide” refers to a cyclized monomer unit based on a compound having a chemical structure H(CHOH)ₙC(═O)(CHOH)ₘH wherein n+m is 4 or 5. Thus, monosaccharides include, but are not limited to, aldohexoses, aldopentoses, ketohexoses, and ketopentoses such as arabinose, lyxose, ribose, xylose, ribulose, xylulose, allose, altrose, galactose, glucose, gulose, idose, mannose, talose, fructose, psicose, sorbose, and tagatose.

The term “monosaccharide analog” refers to a monosaccharide wherein one or more hydroxyl group of the monosaccharide is replaced by another chemical group, such as, but not limited to, a phosphate, an amine, a thiol, or an alkyl group.

The term “amino sugar” refers to a monosaccharide analog wherein one or more hydroxyl group of a monosaccharide is replaced by an amine. An exemplary amino sugar is glucosamine (i.e., 2-deoxy-2-amino-α-D-glucopyranose).

The term “fragment” as used herein with relation to a compound, refers to a compound whose structure is any portion of the structure of the originally named compound that is less than the whole of the originally named compound. Thus, a fragment is smaller than the original compound, but generally retains some or all of the biological activity of the original compound.

“Pharmaceutically acceptable” refers to those compounds, materials, compositions, and/or dosage forms that are, within the scope of sound medical judgment, suitable for contact with the tissues of human beings and animals without excessive toxicity, irritation, allergic response, or other problems or complications commensurate with a reasonable benefit/risk ratio. Thus, in some embodiments, the presently disclosed compounds, materials, compositions, and/or dosage forms are pharmaceutically acceptable for use in humans.

Generally, the term “reducing” refers to methods of treating a pre-existing condition (e.g., edema) by, for example, reducing or alleviating the symptoms or effects of an existing condition, disease, disorder, or injury, to any degree.

“Preventing” refers to methods of keeping a potential future condition, disease, disorder, or injury, or the symptoms thereof, from occurring, to any degree. “Preventing” can refer to methods of reducing or decreasing the effects of a future condition or injury, such that the effects of the future condition or injury are of a lesser magnitude or shorter duration than the effects that would have occurred in the absence of the preventative action, as well as to methods of completely keeping the effects from occurring. Thus, “preventing” refers to prophylactic methods of medical and veterinary treatment.

The term “ligand” refers to a compound that has a binding affinity for a biological receptor, such as a toll-like receptor. The binding of a ligand to a receptor can be reversible or irreversible. In some instances, the binding of ligand to the receptor can cause a biological response or activity (e.g., the biological activity associated with the activation of that receptor). Ligands that bind to a receptor and trigger a biological response can be referred to as “agonists.” Ligands that bind to a receptor but that do not trigger or that prevent a biological response or activity can be referred to as “antagonists.” Agonists or antagonists can compete for binding to a receptor with an endogenous ligand. Agonists and antagonists can be partial or full. For example, binding of a full agonist to a receptor produces the same level of activity as an endogenous ligand for the receptor, while binding of a partial agonist provides only a portion of that level of activity. The efficacy of agonists or antagonists can be expressed as EC₅₀ (half maximal effective concentration) or IC₅₀ (half maximal inhibitory concentration), respectively, for example.

“Edema” refers to an increase in interstitial fluid in a tissue or organ. “Edema” can also refer to an increase in alveolar fluid. Thus, in some embodiments, edema is related to a condition involving increased endothelial permeability. In some embodiments, the edema can be related to ischemia-reperfusion.

“Increased endothelial permeability” refers to increased permeability of blood vessels in an organ or tissue to fluid and/or protein in the blood, resulting in edema, which can occur in a number of clinical scenarios, such as, but not limited to, Adult Respiratory Distress Syndrome (ARDS), Systemic Inflammatory Response Syndrome (SIRS) and in the setting of infection with a variety of bacteria.

“Ischemia” refers to inadequate blood flow to a tissue or organ, which results in the tissue or organ's inability to meet demands for metabolism.

Reperfusion (resumption of blood flow) to the ischemic organ or tissue can lead to the production of excessive amounts of reactive oxygen species (ROS) and reactive nitrogen species (RNS), thus causing oxidative stress which results in a series of events such as alterations in mitochondrial oxidative phosphorylation, depletion of ATP (which also occurs during and as a result of ischemia), an increase in intracellular calcium and activation of protein kinases, phosphatases, proteases, lipases and nucleases leading to loss of cellular function/integrity.

Ischemia reperfusion injury (IRI) refers to an injury which occurs after blood circulation is restarted in a tissue subjected to ischemia (e.g., when an organ is excised by operation and re-attached, as in a transplant or auto-transplant). By way of additional example and not limitation, such injury also occurs when blood circulation is restarted after being stopped for the transplantation of an organ; after a coronary artery is treated with percutaneous transluminal coronary angioplasty (PTCA), stent, or bypass after myocardial infarction; and after administration of a thrombolytic to a stroke patient. Another example is when blood flow to the heart is temporarily stopped for cardiac surgery, often by the concomitant administration of cardioplegia solutions. Another example is interruption of blood flow to a limb for surgery in a bloodless field by an orthopedic surgeon when a tourniquet is inflated on the limb. Such an injury can occur in many tissues, such as kidney, liver, lungs, pancreas, skeletal muscle, soft tissue (e.g., tendons, ligaments, fascia, fibrous tissue, fat, synovial membranes, nerves and blood vessels), and intestines, as well as in the heart and brain. Thus, edema to be treated (e.g., reduced or prevented) by the presently disclosed subject matter can include, but is not limited to, cerebral, retinal, hepatic, renal, pancreatic, spinal cord, mesenteric, limb, intestinal, brain, myocardial, central nervous system, skin, or lung ischemia reperfusion, or a combination thereof. In particular, edema related to ischemia-reperfusion can be treated in organ transplantation.

### II. General Considerations

“Toll-like receptors” or “TLRs” have multiple roles including roles in both embryogenesis and in recognition of pathogen-associated molecular patterns (PAMPs). See Sioud et al., J. Mol. Biol., 364(5), 945-954 (2006); and Janssens and Beyaert, Clin. Microbiol. Rev., 16(4), 637-646 (2003). The TLRs are type I transmembrane proteins containing repeated leucine-rich motifs in their extracellular domains and a cytoplasmic tail that contains a conserved region called the Toll/IL1 receptor (TIR) domain. At least 10 human TLR proteins have been identified, Toll-like receptors 1-10. TLRs play a role in early innate immunity to invading pathogens by sensing microorganisms or noxious environmental agents. These evolutionarily conserved receptors, homologues of the Drosophila Toll gene, recognize highly conserved structural motifs expressed by microbial pathogens (i.e., PAMPs), and sense products of tissue damage by noxious agents or tissue injury, for example dsRNA, hyaluronan fragments, fibronectin and others. PAMPs include various bacterial cell wall components such as lipopolysaccharide (LPS), peptidoglycan (PGN) and lipopeptides, as well as flagellin, bacterial DNA and viral double-stranded RNA. TLRs thus protect mammals from pathogenic organisms, such as viruses, bacteria, parasitic agents, or fungi, and from tissue injury, by generating an “innate immune” response to products of the pathogenic organism. They can additionally protect animals from noxious environmental agents that destroy cells and release dsRNA or other PAMPs that can interact with the TLR.

The innate immune response results in increases in genes encoding several inflammatory cytokines and chemokines, as well as co-stimulatory molecules, and plays a role in the development of antigen-specific adaptive immunity. Stimulation of TLRs by PAMPs initiates a signaling cascade that involves a number of proteins, such as MyD88 and IRAK1. This signaling cascade leads to the activation of the transcription factor NF-κB which induces the secretion of pro-inflammatory cytokines (such as TNFα and IL-1β) and effector cytokines that direct the adaptive immune response. The signaling cascade additionally involves adaptors such as TRIF/TICAM-1 which can signal the IRF-3 pathway to increase Type 1 IFN production, activate Stats, increase IRF-1 gene expression, and activate ISRE's, interferon response factor (IRF) elements. TRL4 is an essential receptor for LPS recognition. In addition, TLR4 has been implicated in the recognition of endogenous ligands, such as heat shock proteins (HSP60 and HSP70), domain A of fibronectins, and oligosaccharides of hyaluronic acid, heparin sulfate and fibrinogen.

Two phases (early and late) have been described in many forms of acute lung injury, dating back to early studies of the effect of endotoxin infusion (see Parker and Brigham, J. Appl. Physiol., 63(3), 1058-1062 (1987)) or activated complement. See Egan et al., J. Surg. Res., 45, 204-214 (1988). The presently disclosed subject matter relates to data that implicates TLR4 on pulmonary microvascular endothelial cells for early development of lung edema due to ischemia-reperfusion. In particular, as described further in the Examples hereinbelow, the presently disclosed subject matter indicates that edema due to ischemia-reperfusion occurs in MyD88−/− mice and that edema due to ischemia-reperfusion occurs irrespective of MAPK and NF-κB activation. This evidence, coupled with the absence of the TRIF pathway in murine endothelial cells (see Harari et al., Circ. Res., 98(9), 1134-1140 (2006)), suggests that edema mediated by TLR4 occurs independent of TLR4-mediated transcriptional events. The presently disclosed subject matter also relates to the finding that CRX-526, a known TLR4 antagonist, prevents edema in models of IRI.

### III. Formula (I)

III.A. Compounds of Formula (I)

In some embodiments, the presently disclosed subject matter relates to the use of compounds in preventing or reducing edema, including edema related to ischemia-reperfusion. In some embodiments, the compound is a lipid

A mimetic comprising a monosaccharide analog. In some embodiments, the monosaccharide analog is an amino sugar. In some embodiments, the amino sugar is glucosamine. In some embodiments, the compound is an aminoalkyl glucosaminide phosphate (AGP) or a pharmaceutically acceptable salt thereof.

In general, AGPs are synthetic (i.e., chemically synthesized) lipid A mimetics and can have a structure of Formula (I):

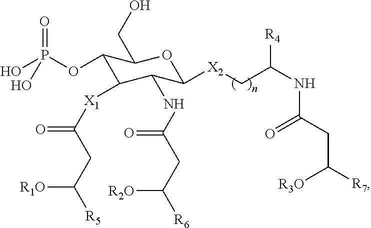

wherein:

n is an integer from 1 to 6;

X₁ is O or S;

X₂ is O or S;

R₁, R₂, and R₃ are independently C₂-C₁₆ acyl;

R₄ is selected from the group consisting of H, hydroxylalkyl, —C(═O)NH₂, and —(CH₂)ₘC(═O)OH, wherein m is an integer from 0 to 2; and

R₅, R₆, and R₇ are independently C₁₀-C₁₂ alkyl, or a pharmaceutically acceptable salt thereof.

Some AGPs act as agonists of TLR4, while others have been reported to inhibit TLR4. See Stöver et al., J. Biol. Chem., 279(6), 4440-4449 (2004). Generally, the inhibitory AGPs include at least one secondary acyl chain (i.e., R₁, R₂, or R₃) that is less than eight carbons. Thus, in some embodiments, at least one of R₁, R₂ and R₃ is -C(═O)R₈, wherein R₈ is C₁-C₆ alkyl (i.e., at least one of R₁, R₂, and R₃ is C₂-C₇ acyl). In some embodiments, at least two of R₁, R₂, and R₃ are C₂-C₇ acyl. In some embodiments, at least one of R₁, R₂ and R₃ is —C(═O)R₈, wherein R₈ is C₅ alkyl. In some embodiments, R₅, R₆, and R₇ are each C₁₀-C₁₂ straight-chain, fully saturated alkyl.

In some embodiments, the compound is CRX-526, i.e., the compound of Formula (I) wherein n is 1; X₁ and X₂ are each O; R₁, R₂ and R₃ are each —C(═O)(CH₂)₄CH₃; R₄ is —C(═O)OH; and R₅, R₆, and R₇ are each —(CH₂)₁₀CH₃, or a pharmaceutically acceptable salt thereof. The synthesis and activity of a variety of AGPs have been previously described. See, e.g., Cluff et al., Infection and Immunity, 73(5), 3044-3052 (2005); Stover et al., J. Biol. Chem., 279(6), 4440-4449 (2004); and references cited therein. See also, U.S. Pat. No. 6,113,918 to Johnson et al.

The compounds of Formula (I) have asymmetric carbon atoms and can therefore exist as enantiomers or diastereomers. Diasteromeric mixtures can be separated into their individual diastereomers on the basis of their physical chemical differences by methods known per se, for example, by chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers and mixtures thereof are considered as part of the presently disclosed subject matter.

III.B. Pharamaceutically Acceptable Salts

The expression “pharmaceutically acceptable salt” as used herein in relation to compounds of the presently disclosed subject matter (e.g., the compounds of Formula (I)) includes pharmaceutically acceptable cationic salts. The expression “pharmaceutically-acceptable cationic salts” is intended to define but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, ethanolamine, diethanolamine, triethanolamine, ethylenediamine, meglumine (N-methylglucamine), benethamine (N-benzylphenethylamine), ethanolamine, diethylamine, piperazine, triethanolamine (2-amino-2-hydroxymethyl-1,3-propanediol) and procaine. In some embodiments, the term “pharmaceutically acceptable salt” as used herein refers to salts that are pharmaceutically acceptable in humans.

Pharmaceutically acceptable salts of the compounds of Formula (I) can be readily prepared by reacting the free acid form of said compounds with an appropriate base, usually one or more equivalent, in a co-solvent. Co-solvents can include, but are not limited to, diethylether, diglyme and acetone. Bases can include, but are not limited to, sodium hydroxide, sodium methoxide, sodium ethoxide, sodium hydride, potassium methoxide, magnesium hydroxide, calcium hydroxide, benzathine, choline, ethanolamine, diethanolamine, piperazine and triethanolamine. The salt is isolated by concentration to dryness or by addition of a non-solvent. In many cases, salts can be prepared by mixing a solution of the acid with a solution of a different salt of the cation (e.g., sodium or potassium ethylhexanoate, magnesium oleate) and employing a co-solvent, as described above, from which the desired cationic salt precipitates, or can be otherwise isolated by concentration.

### IV. Methods of Preventing or Reducing Edema

In some embodiments, the presently disclosed subject matter relates to methods of treating edema. In some embodiments, the presently disclosed subject matter provides a method of preventing or reducing edema in a tissue, the method comprising contacting the tissue with an effective amount of a compound of Formula (I):

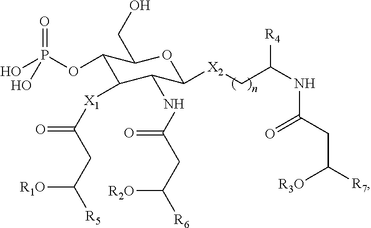

wherein:

n is an integer from 1 to 6;

X₁ is O or S;

X₂ is O or S;

R₁, R₂, and R₃ are independently C₂-C₁₆ acyl;

R₄ is selected from the group consisting of H, hydroxylalkyl, —C(═O)NH₂, and —(CH₂)ₘC(═O)OH, wherein m is an integer from 0 to 2; and

R₅, R₆, and R₇are independently C₁₀-C₁₂ alkyl, or a pharmaceutically acceptable salt thereof.

In some embodiments, at least one of R₁, R₂ and R₃ is —C(═O)R₈, wherein R₈ is C₅ straight-chain, fully saturated alkyl. In some embodiments, R₅, R₆, and R₇ are each C₁₀-C₁₂ straight-chain, fully saturated alkyl.

In some embodiments, n is 1. In some embodiments, X₁ and X₂ are each O. In some embodiments, R₄ is —C(═O)OH. In some embodiments, R₁, R₂, and R₃ are each C₂-C₇ acyl.

In some embodiments, the compound is CRX-526, i.e., the compound of Formula (I) wherein n is 1; X₁ and X₂ are each O; R₁, R₂ and R₃ are each —C(═O)(CH₂)₄CH₃; R₄ is —C(═O)OH; and R₅, R₆, and R₇ are each —(CH₂)₁₀CH₃, or a pharmaceutically acceptable salt thereof.

The edema being prevented or reduced can be related to a number of different causes, including for example, pulmonary edema, inflammation, infection, trauma (e.g., surgery), inhalation of a toxin, a circulatory disorder, or exposure to high altitudes. In some embodiments, the edema is associated with increased endothelial permeability. In some embodiments, the edema to be prevented or reduced is related to (e.g., is the result of or is associated with) ischemia-reperfusion, such as can occur during organ transplantation, tissue transplantation (e.g., during plastic surgery, such as breast reconstruction), autotransplantation (e.g., an autologous tissue or skin graft), other vascularized graft or flap (e.g., muscle graft or myocuteneous flap), pulmonary embolectomy (removal of clotted blood from pulmonary arteries), or pulmonary thromboendarterectomy (surgical removal of organized clot and fibrin from the pulmonary vasculature). In some embodiments, the ischemia-reperfusion is related to myocardial infarction or stroke. In some embodiments, the ischemia-reperfusion is related to cardioplegia (i.e., when cardiac activity is stopped intentionally, such as when perfusion of the heart is interrupted by cross clamping the ascending aorta) during cardiac surgery or to ischemia in skeletal muscle resulting from orthopedic surgery (e.g., when a tourniquet or other device is applied to a limb to reduce blood in the surgical field or otherwise interrupt blood flow).

In some embodiments, the tissue is contacted with an effective amount of the compound prior to a predicted ischemic event (e.g., removal of tissue for organ transplant, cardioplegia, application of a tourniquet, etc) to prevent or reduce damage to the tissue during ischemia or subsequent reperfusion. In some embodiments, the tissue can be contacted with the compound during ischemia. In some embodiments, the tissue can be contacted with the compound after an interval of ischemia (e.g., during reperfusion). In some embodiments, the tissue can be contacted prior to ischemia, during ischemia, after an interval of ischemia, or any combination thereof.

The tissue can comprise skin, bone, bone marrow, brain, cartilage, cornea, skeletal muscle, cardiac muscle, cardiac valve, smooth muscle, blood vessel, a limb or a digit, a kidney or portion thereof, a liver or portion thereof, a heart or portion thereof, a pancreas or a portion thereof, a bowel or portion thereof, or a lung or portion thereof. In some embodiments, the tissue is selected from the group consisting of heart, liver, kidney, brain, small bowel, pancreas, skeletal muscle, skin, and lung tissue. In some embodiments, the lung tissue comprises a lung or a portion thereof (e.g., a lung lobe) provided by a lung transplant donor, wherein the lung tissue is intended for transplant into a lung transplant recipient.

In embodiments involving transplant, a donor or recipient can be a human or a non-human mammal. An organ or tissue transplant donor (e.g., a lung transplant donor) can be living or non-living (i.e., a cadaver). In some embodiments, the donor is a non-heart-beating donor (NHBD). In some embodiments, the donor can be the same individual as the recipient (i.e., in an autologous transplant).

In some embodiments, the tissue could be skeletal muscle, bone and other soft tissues when an interval of ischemia ensues as a result of inflation of a tourniquet to provide a bloodless field for elective orthopedic surgery. In some embodiments, the tissue could be a liver subjected to a Pringle maneuver when blood flow to the liver is temporarily occluded by compression of the portal triad.

Many of the compounds of Formula (I) are expected to be antagonists of TLR 4. Thus, in some embodiments, the compound will be an antagonist of TLR4. In some embodiments, the compound of Formula (I) is an antagonist of TLR2. In some embodiments, the compound is an antagonist of both TLR4 and TLR2.

In some embodiments, the presently disclosed subject matter provides a method of preventing or reducing edema in a subject in need of treatment thereof. In some embodiments, the method comprises administering to the subject an effective amount of a compound of Formula (I) or a pharmaceutically acceptable salt thereof. In some embodiments, the compound is administered to the subject prior to a predicted ischemic event, during ischemia, and/or following an interval of ischemia. The compound can be administered via any suitable route (i.e., oral, intravenously, parenterally, etc.)

In some embodiments, the subject is a mammal. In some embodiments, the subject is a human.

### V. Methods of Preventing or Reducing Edema Related to Ischemia-Reperfusion During Transplantation

In some embodiments, the presently disclosed subject matter provides a method of preventing or reducing edema related to ischemia-reperfusion in a subject of an organ or tissue transplant, the method comprising: providing an organ or tissue for transplant; contacting the organ or tissue with a compound of Formula (I) or a pharmaceutically acceptable salt thereof, thereby providing a treated organ or tissue; and transplanting the treated organ or tissue into a subject in need of said transplant, wherein edema related to ischemia-reperfusion in the subject is prevented or reduced in comparison to edema related to ischemia-reperfusion in a subject of a transplant performed using an organ or tissue untreated with said compound.

In some embodiments, the organ or tissue can be selected from the ‘ group including but not limited to a kidney or portion thereof, a liver or portion thereof, a heart or portion thereof, a retina, a pancreas or a portion thereof, a bowel or portion thereof (e.g., small or large bowel tissue), skeletal muscle tissue, skin tissue, soft tissue, muscle tissue (skeletal muscle or smooth muscle), brain tissue, or a lung or portion thereof. In some embodiments, the organ or tissue is a lung or a portion thereof (e.g., a lung lobe) provided by a lung transplant donor, wherein the lung tissue is intended for transplant into a lung transplant recipient.

An organ or tissue donor or recipient can be a human or a non-human mammal. In some embodiments, the donor and recipient are of the same species. In some embodiments, the donor and the recipient are the same individual. In some embodiments, the donor and recipient are of different species. Thus, the presently disclosed subject matter can be used as part of a xenotransplantation procedure.

The lung (or other organ or tissue) transplant donor can be living or non-living (i.e., a cadaver). In some embodiments, the donor is a non-heart-beating donor (NHBD).

Typically, but not always, a lung transplant donor is the same species as the intended lung transplant recipient. Lung donor selection is generally carried out based on a constellation of clinical findings such as: donor age, smoking history, arterial blood gas, chest radiograph findings, bronchoscopic findings and physical examination of the lung at the time of retrieval. Because the presently disclosed method can reduce or prevent the edema related to ischemia-reperfusion typically related with lung transplant, in some embodiments, lung tissue with slightly reduced function can be considered for transplant (i.e., since less function is being lost during the transplantation procedure).

The contacting can take place by administration of a formulation containing the compound via any suitable route (i.e., oral, intravenous, parenteral, via the airway, etc). The method can further comprise the step of removing said tissue or organ from a donor. Thus, the contacting can take place prior to removing, after removing, or both prior to and after removing. The method can further comprise cold or warm preservation of said tissue or organ. The contacting can take place prior to cold or warm preservation, during cold or warm preservation, or both prior to and during cold or warm preservation. In some embodiments, the contacting can occur via donor inhalation of a pharmaceutical formulation containing the compound. For NHBDs or other donors, the contacting can be performed via the airway. In some embodiments, the contacting can occur via administration of a formulation containing the compound into the pulmonary artery of an ex-vivo perfusion circuit or retrograde via the pulmonary vein. In some embodiments, the contacting can occur in an ex-vivo perfusion circuit or apparatus used to perfuse organs after retrieval from a donor. In some embodiments, the contacting can occur in an ex-vivo ventilation/perfusion circuit or apparatus for refusing and ventilating lungs.

In some embodiments, the compound of Formula (I) is an antagonist of one or both of TLR2 and TLR4. In some embodiments, the compound is an antagonist of both TLR2 and TLR4.

### VI. Pharmaceutical Compositions

As used herein, the term “active compound” can refer to a compound of Formula (I) and to their pharmaceutically acceptable salts. The active compound can be contacted to the tissue through any suitable approach. As used herein, the term “effective amount” refers to an amount of active compound or active compounds which is capable of inhibiting various pathological conditions and sequelae, herein described. The terms “inhibit” or “inhibiting” refers to prohibiting, preventing, treating, alleviating, ameliorating, halting, restraining, reducing, slowing or reversing the progression, or reducing the severity of a pathological condition, such as, but not limited to, a condition related to or resultant from tissue damage (e.g., lung tissue) in subjects who are at risk for edema. As such, the presently disclosed methods of administering active compounds include both medical therapeutic (acute) and/or prophylactic (prevention) administration, as appropriate.

The amount and timing of active compound administered can, of course, be dependent on the subject being treated, on the severity of the affliction, on the manner of administration and on the judgment of the prescribing physician. Thus, because of subject to subject variability, the dosages given below are a guideline and the physician can titrate doses of the compound to achieve the treatment that the physician considers appropriate for the subject. In considering the degree of treatment desired, the physician can balance a variety of factors such as age of the subject, presence of preexisting disease, as well as presence of other diseases. Pharmaceutical formulations can be prepared for oral, intravenous, or aerosol administration as discussed in greater detail below.

The therapeutically effective dosage of any specific active compound, the use of which is within the scope of embodiments described herein, can vary somewhat from compound to compound, and subject to subject, and can depend upon the condition of the subject and the route of delivery. As a general proposition, a dosage from about 0.1 to about 50 mg/kg can have therapeutic efficacy, with all weights being calculated based upon the weight of the active compound, including the cases where a salt is employed. Toxicity concerns at the higher level can restrict intravenous dosages to a lower level, such as up to about 10 mg/kg, with all weights being calculated based on the weight of the active base, including the cases where a salt is employed. A dosage from about 10 mg/kg to about 50 mg/kg can be employed for oral administration. Typically, a dosage from about 0.5 mg/kg to 5 mg/kg can be employed for intramuscular injection. In some embodiments, dosages can be from about 1 μmol/kg to about 50 μmol/kg, or, optionally, between about 22 μmol/kg and about 33 μmol/kg of the compound for intravenous or oral administration.

The in vitro and in vivo assays described herein provide an approach wherein the activities of compounds can be compared. The results of these comparisons are useful for determining dosage levels in mammals, including humans, for inducing protection from edema. Such assays provide for the comparison of activities of the compounds of Formula (I) and other compounds, including other TLR4 and/or TLR2 ligands. The results of these comparisons are useful for determining such dosage levels.

In accordance with the presently disclosed methods, pharmaceutically active compounds as described herein can be administered orally as a solid or as a liquid, or can be administered intramuscularly, intravenously or by inhalation as a solution, suspension, or emulsion. In some embodiments, the compounds or salts also can be administered by inhalation, intravenously, or intramuscularly as a liposomal suspension. When administered through inhalation the active compound or salt can be in the form of a plurality of solid particles or droplets having a particle size from about 0.5 to about 5 microns, and optionally from about 1 to about 2 microns. In some embodiments, the active compounds can be administered in nanoparticle delivery vehicles.

The pharmaceutical formulations can comprise an active compound described herein or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. The dispensing is optionally done by an aseptic method. Sterilized closures can then be placed on the vials and, if desired, the vial contents can be lyophilized.

In addition to the active compounds or their salts (e.g., the compounds of Formula (I)), the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. The antimicrobial preservative is typically employed when the formulation is placed in a vial designed for multi-dose use. The pharmaceutical formulations described herein can be lyophilized using techniques well known in the art.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch (e.g., potato or tapioca starch) and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules. Materials in this connection also include lactose or milk sugar as well as high molecular weight polyethylene glycols. When aqueous suspensions and/or elixirs are desired for oral administration, the compounds of the presently disclosed subject matter can be combined with various sweetening agents, flavoring agents, coloring agents, emulsifying agents and/or suspending agents, as well as such diluents as water, ethanol, propylene glycol, glycerin and various like combinations thereof.

In yet another embodiment of the subject matter described herein, there is provided an injectable, stable, sterile formulation comprising an active compound as described herein, or a salt thereof, in a unit dosage form in a sealed container. The compound or salt is provided in the form of a lyophilizate, which is capable of being reconstituted with a suitable pharmaceutically acceptable carrier to form a liquid formulation suitable for injection thereof into a subject. When the compound or salt is substantially water-insoluble, a sufficient amount of emulsifying agent, which is physiologically acceptable, can be employed in sufficient quantity to emulsify the compound or salt in an aqueous carrier. Particularly useful emulsifying agents include phosphatidyl cholines and lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound can be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques. The liposomal formulations comprising the active compounds disclosed herein can be lyophilized to produce a lyophilizate, which can be reconstituted with a pharmaceutically acceptable carrier, such as water, to regenerate a liposomal suspension.

Pharmaceutical formulations also are provided which are suitable for administration as an aerosol by inhalation. These formulations comprise a solution or suspension of a desired compound described herein or a salt thereof, or a plurality of solid particles of the compound or salt. The desired formulation can be placed in a small chamber and nebulized. Nebulization can be accomplished by compressed air or by ultrasonic energy to form a plurality of liquid droplets or solid particles comprising the compounds or salts. The liquid droplets or solid particles should have a particle size in the range of about 0.5 to about 10 microns, and optionally from about 0.5 to about 5 microns. The solid particles can be obtained by processing the solid compound or a salt thereof, in any appropriate manner known in the art, such as by micronization. Optionally, the size of the solid particles or droplets can be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation can comprise a water-soluble active compound in a carrier that comprises water. A surfactant can be present, which lowers the surface tension of the formulation sufficiently to result in the formation of droplets within the desired size range when subjected to nebulization.

As indicated, both water-soluble and water-insoluble active compounds are provided. As used herein, the term “water-soluble” is meant to define any composition that is soluble in water in an amount. of about 50 mg/mL, or greater. Also, as used herein, the term “water-insoluble” is meant to define any composition that has a solubility in water of less than about 20 mg/mL. In some embodiments, water-soluble compounds or salts can be desirable whereas in other embodiments water-insoluble compounds or salts likewise can be desirable.

In one mode of administration, the compounds of the presently disclosed subject matter can be administered just prior to a surgery (e.g., within twenty-four hours before surgery, for example, cardiac surgery or transplant surgery), during and/or subsequent to surgery (e.g., within twenty-four hours after surgery) where there is risk of ischemia. In another mode of administration, the active compounds are administered with an initial loading dose (e.g., bolus injection or infusion) prior to surgery followed by a constant infusion prior to, during and post surgery. The active compounds can also be administered in a chronic daily mode.

Methods of preparing various pharmaceutical compositions and with a certain amount of active ingredient are known, or can be determined, in light of this disclosure, by those skilled in this art. For examples of methods of preparing pharmaceutical compositions, see Remington's Pharmaceutical Sciences, Mack Publishing Company, Easter, Pa., 16th Edition (1980). Pharmaceutical compositions according to the presently disclosed subject matter can contain, for example, 0.0001%-95% of the active compound(s). In any event, the composition or formulation to be administered can contain a quantity of an active compound(s) in an amount effective to treat the disease/condition of the subject being treated.

In some embodiments, the methods of the presently disclosed subject matter can be used to prevent or reduce edema related to ischemia-reperfusion in extracorporeal tissue or organs or in tissue or organs that are being transplanted from a tissue or organ donor into a transplant recipient. Extracorporeal tissue or organs are tissue or organs not in an individual (also termed ex vivo). For tissue and organ transplantation, donor tissue and organs removed are also subjected to ischemia-reperfusion during harvesting, while in transit and following transplantation into a recipient. The presently disclosed methods can be used to improve the function of a transplantable tissue or organ by, for example, supplementing solutions used to maintain or preserve transplantable tissues or organs. For example, the methods and compositions can be used to bathe the transplantable tissue or organ during transport or can be placed in contact with the transplantable tissue or organ prior to, during or after transplantation. In some embodiments, formulations of the presently disclosed subject matter can be contacted to a tissue or organ while the tissue or organ is present in the donor.

Solutions of the presently disclosed subject matter can be used in perfusion devices (e.g., ex vivo perfusion circuits). A perfusion device as used herein is any mechanical device that be used to infuse a specific organ or the systemic circulation with a solution comprising a compound or composition. Such a device can contain one or more reservoirs. The device can include a tube, catheter, or cannula leading from the reservoir that can be inserted into an organ, vein or artery. The device can be an electromechanical device having electric pumps and devices for controlling the temperature, rate or volume of delivery of the solution. In certain embodiments, the device is programmable so that the one or more solutions are delivered in an appropriate temperature, rate or volume for a particular clinical situation, weight of the organ, or size of the organ (e.g., cardiopulmonary bypass surgery vs. kidney transplant vs. liver transplant). Accordingly, in some embodiments, the presently disclosed subject matter relates to preservation solutions for ex vivo organs or tissues. In some embodiments, the preservation solution comprises a compound of Formula (I). In some embodiments, the compound of Formula (I) is CRX-526. In some embodiments, the compound is an antagonist of both TLR2 and TLR4.

### VII. Subjects

In some embodiments, the subject treated in the presently disclosed subject matter is desirably a human subject, although it is to be understood the methods described herein are effective with respect to all vertebrate species, which are intended to be included in the term “subject.” The methods described herein are particularly useful in the treatment and/or prevention of edema such as but not limited to edema related to ischemia-reperfusion in warm-blooded vertebrates. Thus, the methods can be used as treatment for mammals and birds. In some embodiments, the subject of the presently disclosed method is an organ transplant recipient.

More particularly, provided herein is the treatment of mammals, such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided herein is the treatment of birds, including the treatment of those kinds of birds that are endangered, kept in zoos or as pets, as well as fowl, and more particularly domesticated fowl, i.e., poultry, such as turkeys, chickens, ducks, geese, guinea fowl, and the like, as they also are of economical importance to humans. Thus, embodiments of the methods described herein include the treatment of livestock, including, but not limited to, domesticated swine (pigs and hogs), ruminants, horses, poultry, and the like.

### EXAMPLES

The following Examples provide illustrative embodiments. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### General Methods

Male C3H/HeJ, C3H/OuJ, TLR4−/− and C57BL/6J mice were purchased from Jackson Laboratories (Bar Harbor, Me., United States of America) and MyD88−/− mice were provided by Dr. Shizuo Akira (Osaka University, Osaka, Japan). See also, Adachi, O., et al., Immunity, 9, 143-150 (1998). Mice were maintained in a pathogen-free facility until they weighed 25-30 grams and were 8-10 weeks old. Reagents were from Sigma (St. Louis, Mo., United States of America) unless specified.

### Surgical Model of Murine Lung IRI and Assessment of Barrier Function

Mice were anesthetized with ketamine (0.1 mg/gm body weight) and xylazine (0.01 mg/gm) interperitoneally, followed by one-third of the initial dose hourly. Tracheotomy allowed mechanical ventilation (with a tidal volume of 0.4 mL, respiratory rate 120/min, I/E 0.4, PEEP 3 cm H₂O, FiO₂, 1.0) using a Columbus Instruments Ventilator CIV-101 (Columbus Instruments, Columbus, Ohio, United States of America). The right jugular vein was cannulated for infusion of albumin 2.5% in 0.9% saline, 450 μL/hour by syringe pump (Medfusion 2010i; Medex, Carlsbad, California, United States of America) to maintain hydration. Rectal temperature was monitored and maintained with a heating pad. The left pulmonary hilum was occluded for 1 hour with a microvascular clamp through a left thoracotomy. Reperfusion began with removal of the clamp. Animals were sacrificed at intervals ranging from 15 minutes to 3 hours by cardiectomy, and both lungs were excised. The apical portion of each lung was excised and immediately weighed, then desiccated in a 60° C. oven for 48 hours and re-weighed to determine wet to dry weight ratio (W/D). Remaining lung tissue was flash frozen in liquid nitrogen and stored at −80° C. Lungs excised immediately after sacrifice served as controls.

Extravascular Albumin Extravasation with Evans Blue Dye (EBD)

Extravascular albumin extravasation after 1 hour IRI was assessed by the EBD technique as previously described. See Saria et al., J. Neurosci Methods, 8(1), 41-49 (1983). After occlusion of the left hilum, 30 mg/kg of EBD dissolved in 250 μL of 0.9% saline solution were injected into the right jugular vein. After 1 hour of reperfusion, the chest was opened through a median sternotomy, the mice were euthanized by right ventriculotomy, the pulmonary trunk cannulated with an 18 gauge angio-catheter, and the left atrial appendage amputated. Both lungs were flushed with normal saline to remove intravascular EBD, excised and weighed. The lung tissue was suspended in formamide (100 mg lung tissue/1 mL formamide; Roche Diagnostics, Indianapolis, Ind., United States of America) and incubated for 24 hours at 50° C. Specimens were then centrifuged (13,000 g×30 minutes), and 50 μL of supernatant were placed in 96-well plates for colorimetric assessment in a μQuant spectrophotometer (Bio-Tek Instruments, Inc., Winooski, Vermont, United States of America) at 620 nm. Relative optical density values were normalized by the weight of the samples.

### Inflation Fixation for Histology

After 60 or 180 minutes of IRI (n=4/strain/group), lung blocks were inflation-fixed through the trachea with 4% buffered paraformaldehyde at a constant pressure of 25 cm H₂O for 24 hours at room temperature, then embedded in paraffin. Five micron sections were stained with hematoxylin and eosin. Lungs from animals sacrificed immediately after tracheotomy (n=4/strain) served as controls.

### Immunostaining for NF-κB Translocation

Immunohistochemical staining of inflation fixed lung tissue was performed using a rabbit polyclonal p65 antibody (ab 31481; Abcam plc, Cambridge, United Kingdom) at a 1:100 dilution. Samples were sectioned at 5 μm, dried overnight and baked at 60° C. for one hour. Sections were deparaffinized and epitope retrieval was done with 6.0 pH Citra Antigen Retrieval Buffer (Dakocytomation, Carpinteria, Calif., United States of America) for 30 min at 100° C. Background was blocked using a Peroxidase block, a serum-free Protein Block, and an Avidin/Biotin block (Dakocytomation, Carpinteria, Calif., United States of America). Sections were incubated with the primary antibody p65 overnight at 4° C. Detection was completed with the LSAB+secondary antibody along with a DAB chromagen for visualization (Dakocytomation, Carpinteria, Calif., United States of America). No counterstain was applied. Slides were scored for p65 nuclear staining by a pathologist blinded to specimen group and graded as 1+(mild, some nuclear staining evident), 2+(moderate, some intense staining, but not consistent) or 3+(dark consistent staining of virtually all nuclei).

### Western Blotting and Densitometry

Protein concentration measurement and immunoblotting were performed as previously described. See Wu et al., Respir Res, 6(1), 26 (2005). Briefly, frozen lung tissue was suspended in 10 μL/mg ice-cold RIPA lysis buffer (100 mM Tris-HCl pH 8.0, 100 mM NaCl, 5 mM NaF, 2 mM EDTA, 1% NP-40, 1 mM Na₃VO₄, 100 μM TPCK, 1 μM pepstatin A, 2 μM leupeptin, 1 mM PMSF, 100 μM quercetin), Dounce homogenized, and centrifuged at 13,200 rpm for 10 minutes at 4° C. to remove insoluble material. Supernatant protein concentrations were determined using the Coomassie Protein Assay Reagent (Pierce Biotechnology, Rockford, Ill., United States of America). After addition of β-mercaptoethanol (5%) and tracking dye, samples were denatured, and equivalent amounts of protein were resolved by SDS-PAGE (10% tris-glycine or 4%-12% bis-tris gels; lnvitrogen, Carlsbad, Calif., United States of America) and transferred onto lmmobilon-P membranes (Millipore Corp., Billerica, Mass., United States of America). Blots were blocked in TBS with 0.1% Tween-20 and 5% nonfat dry milk powder for 1 hour, incubated with primary and then secondary antibodies, followed by chemiluminescent detection of peroxidase (Millipore Corp., Billerica, Mass., United States of America). Antibodies against phosphorylated or total JNK, p38, ERK, and IκBα were purchased from Cell Signaling Technology (Beverly, Mass., United States of America). Films were scanned at 600 dpi in 16-bit grayscale on an Epson Precision 4180 flatbed scanner (Epson America, Inc., Long Beach, Calif., United States of America). Densitometry was performed using METAMORPH® software (MDS Analytical Technologies, Inc., Sunnyvale, Calif., United States of America).

### Bone Marrow Transplant (BMT)

Chimeric mice were generated by BMT using procedures described previously. See Schwaller et al., Embo J, 17(18), 5321-5333 (1998). Recipient mice were exposed to 12 Gy lethal irradiation (Gammacell 40¹³⁷ Cs γ-irradiation source; Nordion, Ottawa, Canada), delivered in 2 doses separated by 4 hours (700 cGy, then 500 cGy). Bone marrow was obtained from donor mice by flushing their femurs and tibias with medium (Roswell Park Memorial Institute (RPMI) buffer+10% fetal bovine serum (FBS)+100 units Heparin+1 M HEPES). Harvested marrow cells were passed through a 0.2 μm filter, enumerated and resuspended to a concentration of 10⁶ cells in 200 μL of sterile PBS +10% FBS. Marrow cells were then injected retro-orbitally into recipients immediately after they received the second dose of γ-radiation. Recipient mice were maintained in sterile microisolator cages for 12 weeks to allow full humoral reconstitution.

Four sets of chimeras were created to produce mice with functional TLR4 (+) on parenchymal cells (P) or myeloid cells (M). HeJ mice had marrow reconstituted from OuJ donors (P−M+); while OuJ mice had marrow reconstituted from HeJ mice (P+M−). “Control” chimeras were generated by reconstituting marrow from the same strains (P−M−) and (P+M+).

### Determination of Viability for Cell Culture Experiments

In separate experiments performed in triplicate, HMVECs grown to confluence on P35 dishes underwent simulated IRI. At the same time points, cells and cell culture media or Ringer's lactate were assessed for lactate dehydrogenase (LDH) activity using the CytoTox96 Non-Radioactive Cytotoxicity Assay (Promega, Madison, Wis., United States of America) following the manufacturer's instructions. Control samples were also taken at time zero and 24 hours to assess cell viability apart from the experimental model. Culture medium and Ringer's lactate were used as background controls to normalize the absorbance value from the other samples. Cytotoxicity was calculated as media LDH activity divided by total LDH activity (cell pellet plus media). Viability was the inverse and expressed as percent viability at each time point. Bronchoalveolar Lavage (BAL) and Alveolar Macrophage (AM) Cell Culture

AMs from HeJ and OuJ mice were harvested by BAL 120 days after BMT. The trachea was cannulated with a tailored 18 gauge catheter (Becton Dickinson, Sandy, Utah, United States of America). BAL was performed by slow tracheal delivery of 4 aliquots (35 μL×body weight in grams) of pre-warmed, sterile, endotoxin-, calcium-, and magnesium-free PBS with 0.2 mM EGTA. Lavage fluid was withdrawn by gentle suction, pooled for each mouse, and centrifuged at 250 g for 5 minutes. Cells were resuspended in RPMI 1640 (Gibco BRL, Rockville, Md., United States of America) containing 10% heat-inactivated FBS (Atlanta Biologicals, Lawrenceville, Ga.), penicillin G (100 U/mL), and streptomycin (100 μg/mL). Viability was consistently >95% by trypan blue exclusion. Cells were plated at 20,000 per well in 96-well plates. After 2 hours of incubation, plates were washed with PBS to remove non-adherent cells. Adherent AMs were cultured in RPMI 1640 at 37° C. in a humidified incubator with 5% CO₂.

### NF-κB Reporter Assay

Recombinant, first generation E1, E3-deleted adenovirus serotype 5 vectors were prepared (see Sanlioglu et al., J. Biol. Chem., 276, 30188-30198 (2001)) and HMVECs and AMs were transfected as previously described for epithelial cells. See Wu et al., Respir. Res., 6, 26 (2005).

### Statistical Analysis

All data are reported as mean±SEM. Groups were compared by ANOVA with Tukey's post hoc test using STATISTICA® (StatSoft, Inc., Tulsa, Okla., United States of America) or by paired or unpaired t tests.

### Example 2

### TLR4 Mediation of Pulmonary Edema Related to Ischemia-Reperfusion

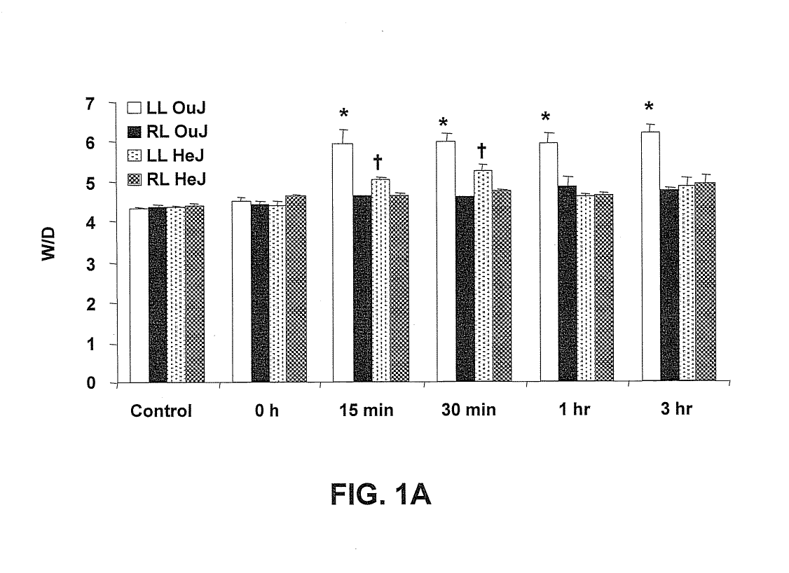

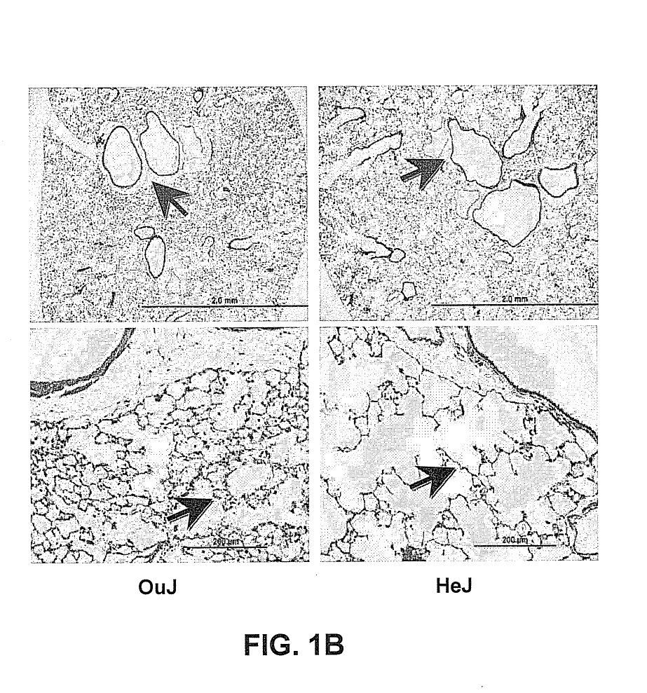

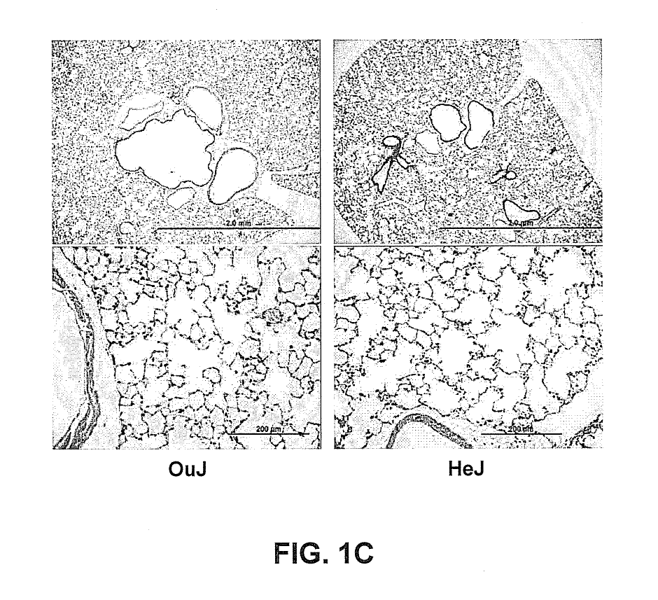

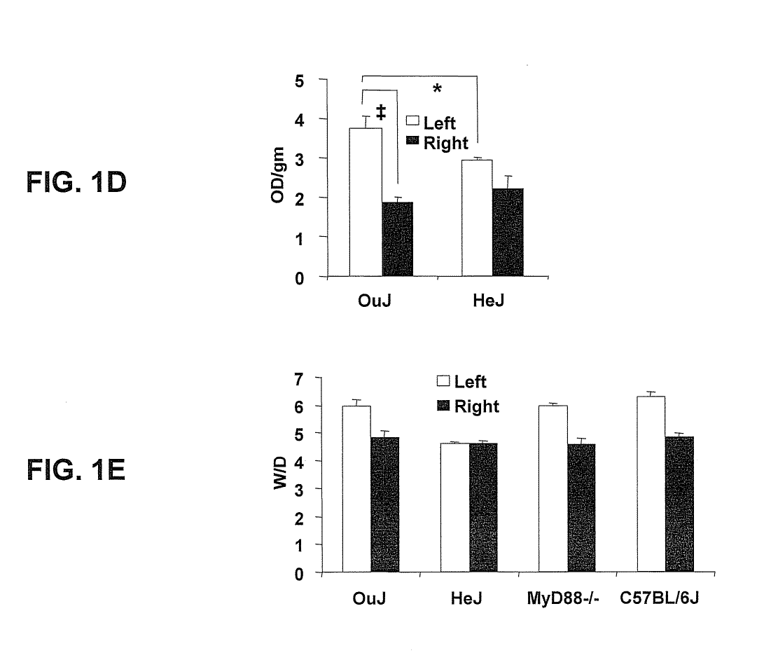

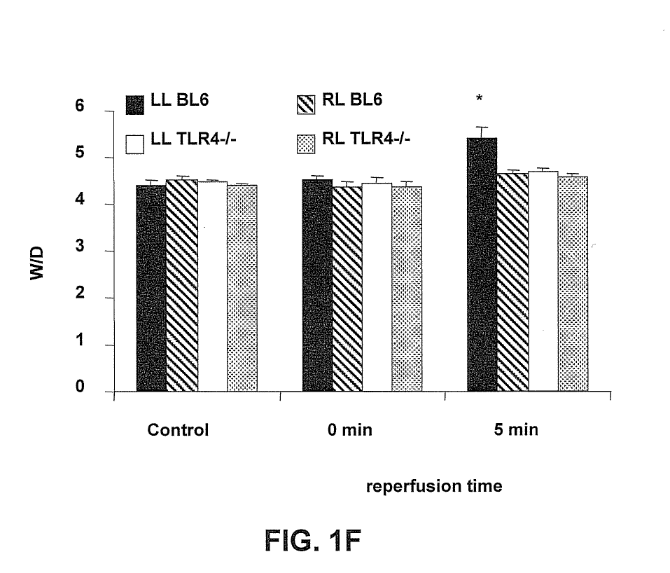

The influence of TLR4 on ischemia-reperfusion-related pulmonary edema was studied by comparing post-ischemia fluid accumulation in the lungs of TLR4 sufficient (OuJ) and deficient (HeJ) mice. As shown in FIG. 1A, reperfusion of left lungs rendered ischemic by 1 hour of hilar clamping induced early, pronounced fluid accumulation (as manifested by elevated W/D) in the left lung of OuJ mice within 15 minutes of reperfusion that persisted out to 3 hours of reperfusion. In contrast, HeJ mice experienced significantly less edema following 15 and 30 minutes reperfusion, and demonstrated earlier recovery. W/D in the HeJ mice after 1 and 3 hours reperfusion was normal.

Additionally, as shown in FIG. 1B, there was more perivascular and alveolar wall edema in inflation-fixed left lungs from OuJ mice reperfused for 3 hours than in lungs from HeJ mice. As shown in FIG. 1C, however, there was no histologic difference in interstitial edema between mouse strains after 1 hour reperfusion. Four HeJ specimens after 3 hours reperfusion and all eight lung specimens studied after one hour were judged to be normal and not different from four control specimens (2 HeJ and 2 OuJ) by a masked observer. Without being bound to any one theory, it is postulated that the increased interstitial edema in OuJ lungs after 3 hours reperfusion was due to rapid alveolar flooding rendered undetectable by inflation fixation 60 minutes after reperfusion. Consistent with this hypothesis, left lungs from OuJ mice had increased EBD content (a measure of microvascular permeability to albumin (see Saria et al., J Neurosci Methods, 8(1), 41-49 (1983))) compared to left lungs from HeJ mice and to right lungs from both strains. See FIG. 1D. Thus, the difference in W/D appears to be due to alveolar flooding occurring early in OuJ mice compared to HeJ mice, with later absorption of the fluid into the alveolar walls and interstitium.

TLR4 signaling downstream of receptor activation involves recruitment of adapter proteins including myeloid differentiation primary response gene 88 (MyD88) and TIR-domain-containing adaptor-inducing interferon-β (TRIF). See O'Neill et al., Nat Rev Immunol, 7(5), 353-364 (2007). Because TRIF is not present in murine endothelial cells (see Harari et al., Circ Res, 98(9), 1134-1140 (2006)), MyD88 signaling is the key adapter downstream of TLR4 in these cells. When MyD88-deficient (MyD88−/−) mice were subjected to 1 hour of IRI, equivalent edema developed in the lungs of the MyD88−/− mice as in those from OuJ mice and C57BL/6J mice, the background strain for MyD88−/− mice. See FIG. 1E. Thus, it appears that TLR4-mediated lung edema due to ischemia-reperfusion is independent of downstream signaling via the MyD88 adapter. To confirm that early edema was due to TLR4, the experiments were repeated in TLR4−/− mice and compared to C57BL/6J mice, the background strain used for the TLR4−/− mice. TLR4−/− mice develop significantly less edema compared to C57BL/6J mice after one hour hilar clamping and reperfusion at 15, 30, and 60 reperfusion. As shown in FIG. 1F, edema appears quickly (within about 5 minutes of reperfusion) in C57BL/67 mice, but not in TLR4−/− mice.

### Example 3

### TLR4 Mediates Early MAPK and NF-κB Activation Due to Lung IRI

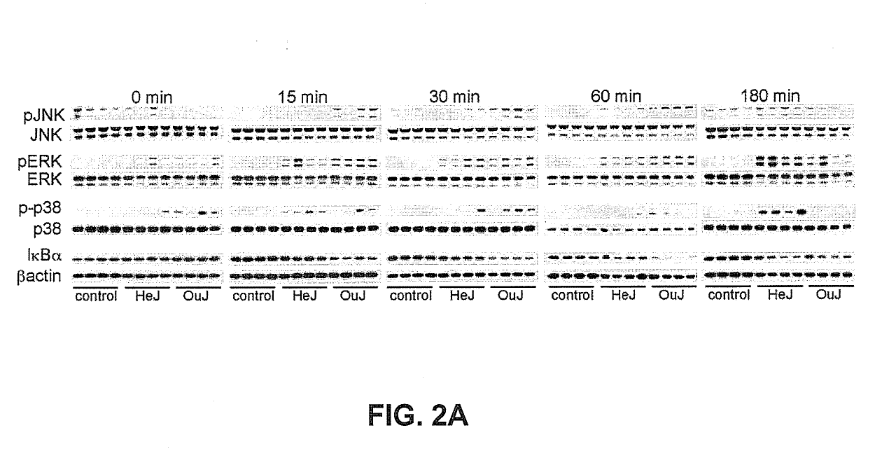

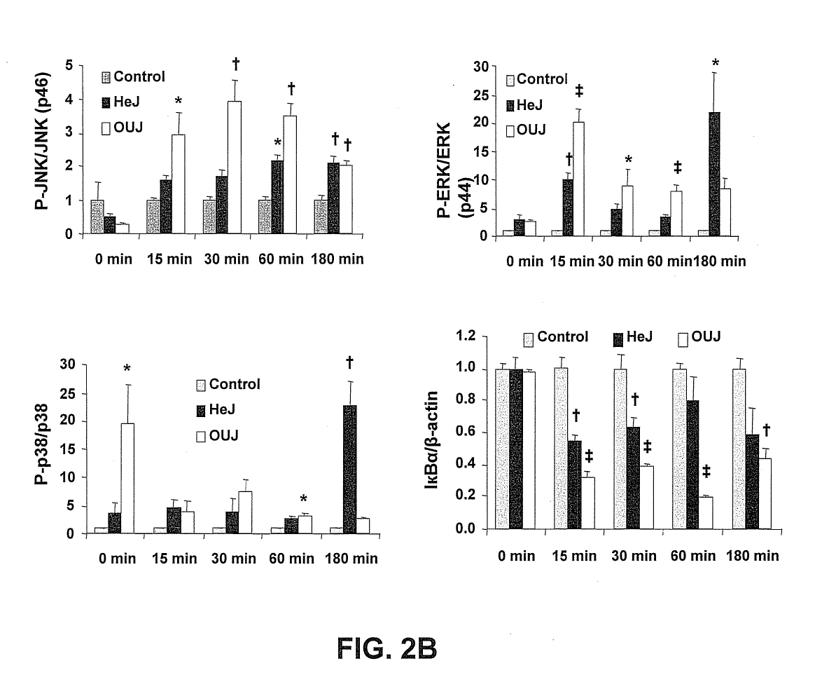

Protein concentration measurements indicated that in addition to a role in early edema formation after lung ischemia, functioning TLR4 mediates early activation of signaling pathways associated with inflammation. As shown in FIGS. 2A and 2B, functioning TLR4 in OuJ mice resulted in early phosphorylation of p38 (observed during ischemia), early phosphorylation of ERK and JNK, and early activation of NF-κB following reperfusion. In comparison, the TLR4-deficient HeJ mice showed delayed or reduced p38, ERK, NF-κB and JNK activation. However, some degree of MAPK and NF-κB activation was observed in HeJ mice, implying involvement of alternative activation pathways other than TLR4.

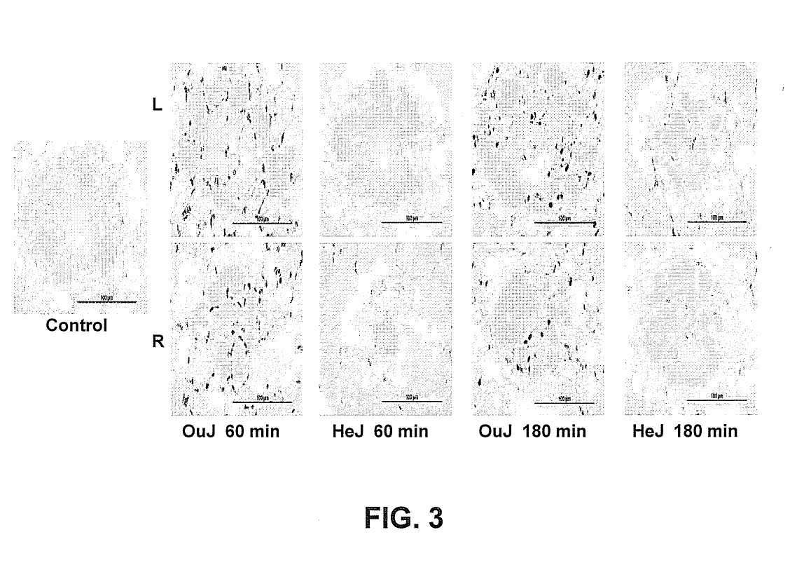

As shown in FIG. 3, immunostaining for the p65 component of NF-κB showed minimal nuclear localization in the lungs of control (freshly sacrificed) mice, while marked nuclear staining (where staining was graded 3+) was observed in the 60 min reperfused samples from TLR4-sufficient (OuJ) mice compared to TLR4-deficient (HeJ) mice (where staining was graded 1-2+). The immunostaining intensity complemented the IκBα degradation seen in FIGS. 2A and 2B, except that IκBα levels appear equivalent in HeJ and OuJ strains at 180 min reperfusion despite more p65 staining in OuJ animals at 180 min reperfusion. This suggests some recovery of IκBα protein in OuJ mice 180 min post-reperfusion. Surprisingly, p65 staining was the same for right and left lungs, implying that NF-κB was activated in the right (non-ischemic) lung to the same extent at the same reperfusion times despite the lack of edema in the right lung. Thus, it appears that NF-KB activation is not necessarily associated with edema development. p38 activation was apparent in left lungs from HeJ mice 3 hours after reperfusion, with normal W/D. Taken together with the rapidity of development, the acute phase pulmonary edema in this model does not appear to be due to MAPK or NF-κB activation.

### Example 4

### TLR4 on Lung Parenchymal Cells Versus Bone-Marrow Derived Cells

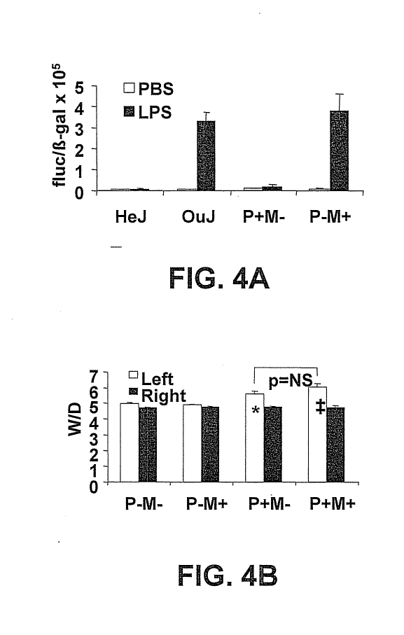

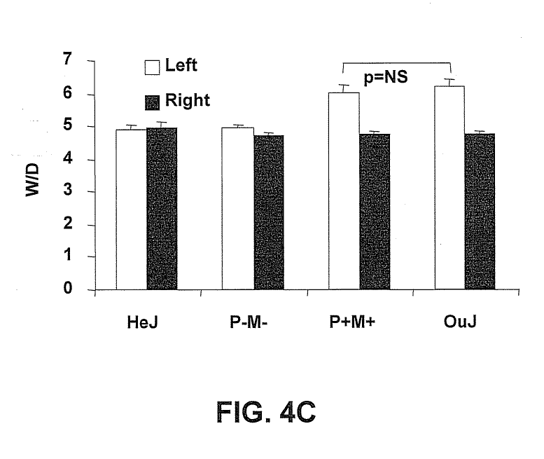

To determine the importance of functioning TLR4 on lung parenchymal versus bone marrow-derived cells, particularly alveolar macrophages (AMs), chimeric mice were created as described in Example 1 by lethally irradiating mice of each strain (OuJ and HeJ) and re-constituting bone marrow by bone marrow transplant (BMT). As shown in FIG. 4A, lipopolysaccharide (LPS) stimulation resulted in an approximately 60-fold increase in luciferase activity in both native OuJ AMs and in AMs retrieved from chimeric strain P-M+, the chimera with TLR4 expressing marrow-derived cells and TLR4 non-expressing parenchymal cells. Thus, it appears that replacement of AMs in chimeric animals was virtually complete 12 weeks after BMT. AMs retrieved from irradiated mice reconstituted with the same strain marrow behaved in the same manner.

Following 3 hours of ischemia-reperfusion, P+M− chimeric animals developed significant increase in W/D. See FIG. 4B. However, even if AMs had functioning TLR4 (P−M+), W/D was not elevated. Thus, edema is apparent only when functional TLR4 is present on lung parenchymal cells (P), whether or not functioning TLR4 is present on myeloid cells (M). While functioning TLR4 on AMs is not sufficient for development of edema, it is possible that it amplfies edema in mice with functioning TLR4 on lung parenchymal cells, as it was observed that W/D was slightly higher in P+M+ animals compared to P+M− animals. However, this difference was not statistically significant.

As shown in FIG. 4C, chimerib controls (OuJ into OuJ and HeJ into HeJ) showed no difference in pulmonary edema formation compared to non-irradiated strains, demonstrating that lethal irradiation had no impact on development of edema due to ischemia-reperfusion. AMs from these “control chimerics” had the same response to LPS as AMs from the native strains (data not shown).

Thus, early edema formation due to ischemia-reperfusion is attributable to functioning TLR4 on lung parenchymal cells, but functioning TLR4 on myeloid cells is not critical to early ischemia-reperfusion-related edema. The presently disclosed data suggests that ischemia-reperfusion-induced pulmonary edema is due to increased capillary leak very early after reperfusion.

### Example 5

### Competitive Inhibitor of TLR4 Prevents Pulmonary Edema Due to Ischemia-Reperfusion

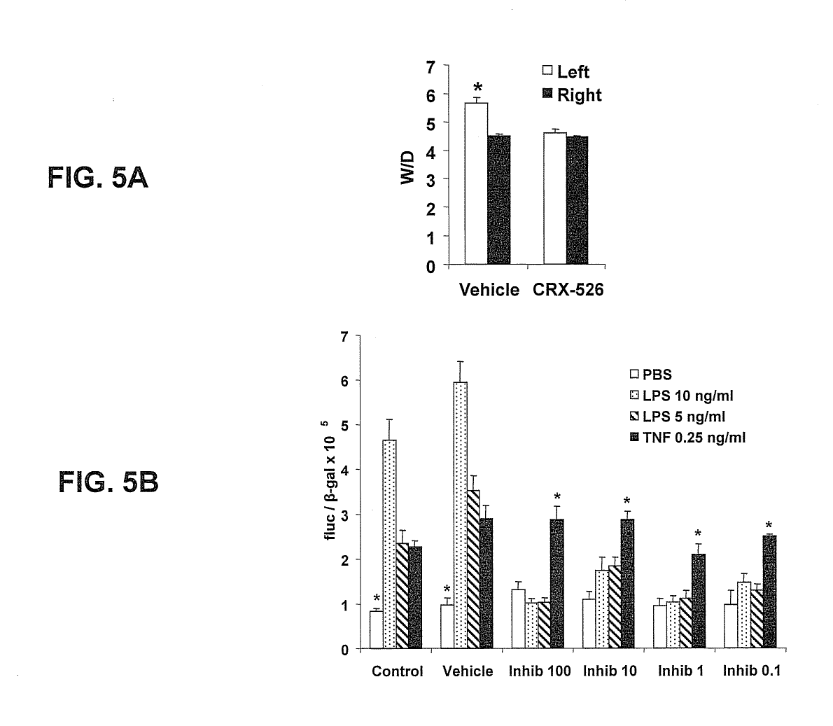

The competitive TLR4 inhibitor CRX-526 administered intravenously over 30 minutes (10 g in 200 μL of saline) to OuJ mice starting 60 minutes before left hilar clamping prevented edema following 1 hour of ischemia-reperfusion. See FIG. 5A. CRX-526 also prevented NF-κB activation in cultured human pulmonary microvascular endothelial cells (HMVECs) exposed to LPS. See FIG. 5B. The TLR4 inhibitor appeared to have no impact on TNF stimulation of NF-KB activation.

### Example 6

### TLR2 and lschemia-Reperfusion

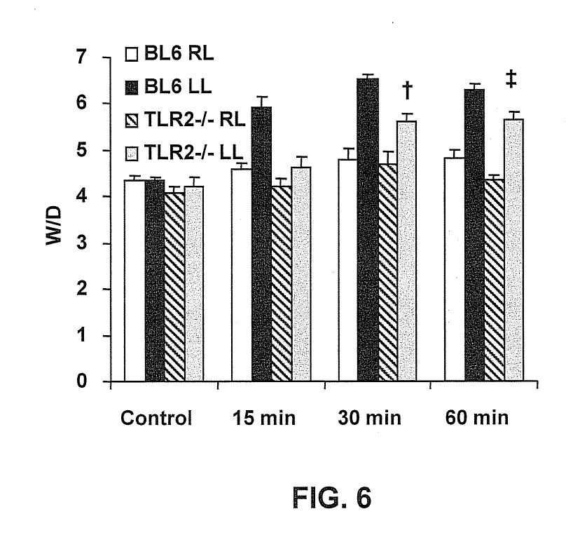

The effect of functioning TLR2 on ischemia-reperfusion-related edema was studied by comparing W/D after 1 hour hilar occlusion and reperfusion in TLR2-deficient (TLR2 −/−) mice bred on the C57BL/6J (BL6) background strain with W/D in TLR2-sufficient BL6 mice. As shown in FIG. 6, TLR2−/− mice develop edema due to IRI, but later than the BL6 mice. W/D in the left lung of the TLR2−/− mice was normal after 15 minutes of reperfusion, while W/D in the BL/6 mice was elevated.

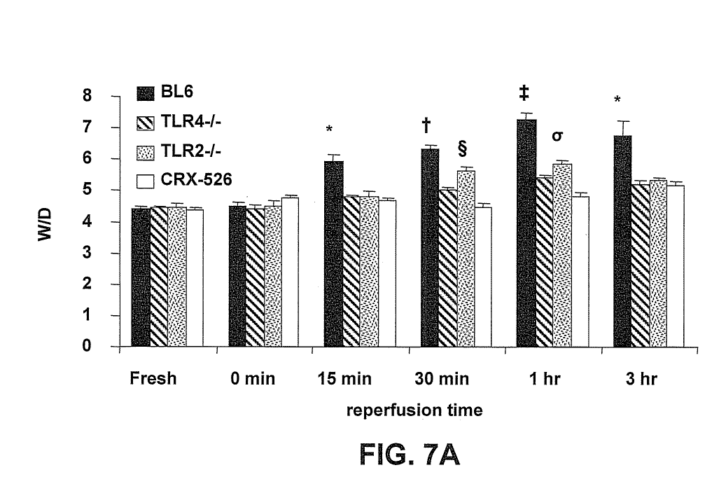

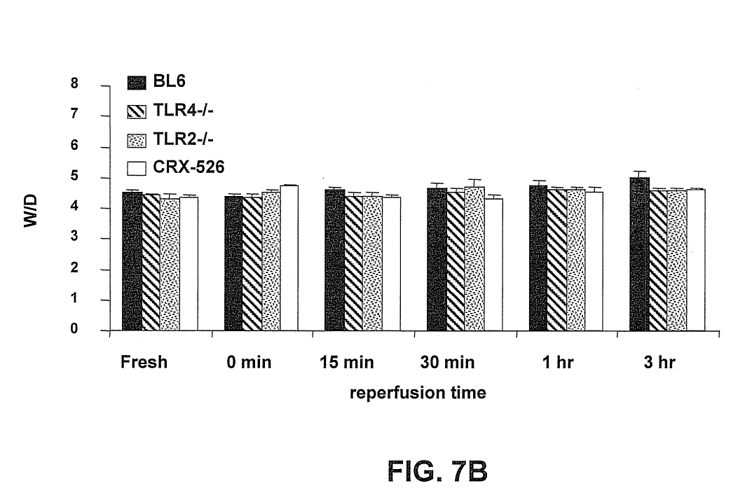

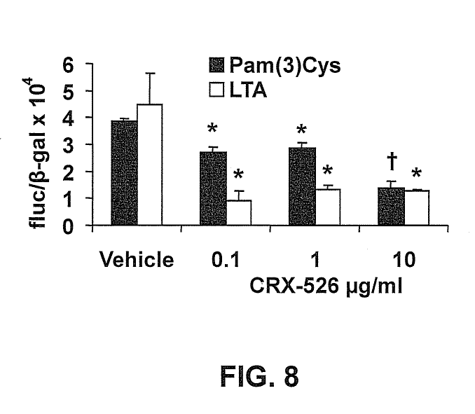

A further study was performed to compare the effects of pre-treatment with a TLR4 inhibitor. BL6 mice were pretreated with CRX-526 (10 μg) administered over a 30 minute time period starting 60 min prior to left hilar clamping for 1 hour. These mice develop little if any edema for up to 180 min of reperfusion. See FIGS. 7A and 7B. The CRX-526 treatment appears to produce W/D similar to the added effects of TLR4 and TLR2 deficiency, particularly after 30 and 60 min reperfusion. As shown in FIG. 8, studies in transfected HMVECs indicated that CRX-526 can reduce NF-κB activation stimulated by TLR2 ligands.

Taken altogether, the presently disclosed data suggests that the effects of CRX-526 on the reduction of ischemia-reperfusion-related edema are due to more than merely its ability to inhibit TLR4.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method of preventing or reducing edema in a tissue, the method comprising contacting the tissue with an effective amount of a compound of Formula (I): 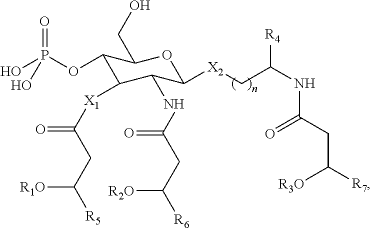
wherein:
n is an integer from 1 to 6;
X₁ is O or S;
X₂ is O or S;
R₁, R₂, and R₃ are independently C₂-C₁₆ acyl, wherein at least one of R₁, R₂, and R₃ is C₂-C₇ acyl;
R₄ is selected from the group consisting of H, hydroxylalkyl, —C(═O)NH₂, and —(CH₂)ₘC(═O)OH, wherein m is an integer from 0 to 2; and
R₅, R₆, and R₇ are independently C₁₀-C₁₂ alkyl, or a pharmaceutically acceptable salt thereof.

2. The method of claim 1, wherein n is 1.

3. The method of claim 1, wherein X₁ and X₂ are each O.

4. The method of claim 1, wherein R₄ is —C(═O)OH.

5. The method of claim 1, wherein R₁, R₂, and R₃ are each C₂-C₇ acyl.

6. The method of claim 1, wherein the compound of Formula (I) is a compound wherein:
n is 1;
X₁ is O;
X₂ is O;
R₁, R₂ and R₃ are each —C(═O)(CH₂)₄CH₃;
R₄ is —C(═O)OH; and
R₅, R₆, and R₇ are each —(CH₂)₁₀CH₃, or
a pharmaceutically acceptable salt thereof.

7. The method of claim 1, wherein the edema is related to ischemia-reperfusion.

8. The method of claim 7, wherein the ischemia-reperfusion is related to myocardial infarction or stroke.

9. The method of claim 7, wherein the ischemia-reperfusion is related to cardioplegia during cardiac surgery or to ischemia in skeletal muscle resulting from orthopedic surgery.

10. The method of claim 7, wherein the ischemia-reperfusion is related to organ or tissue transplant.

11. The method of claim 10, wherein the tissue transplant is a skin transplant, a muscle transplant or a soft tissue transplant.

12. The method of claim 11, wherein the tissue transplant is an autologous tissue transplant.

13. The method of claim 7, wherein contacting the tissue with an effective amount of the compound occurs prior to ischemia, during ischemia, or after an interval of ischemia.

14. The method of claim 1, wherein the tissue is selected from the group consisting of heart, liver, kidney, brain, small bowel, large bowel, pancreas, skeletal muscle, skin, soft tissue, and lung tissue.

15. The method of claim 14, wherein the tissue is from an organ donor.

16. The method of claim 15, wherein the tissue is lung tissue from a lung transplant donor.

17. The method of claim 16, wherein the lung transplant donor is a human lung transplant donor.

18. The method of claim 15, wherein the organ donor is a non-heart-beating donor.

19. The method of claim 1, wherein the compound is an antagonist of one or both of Toll-like receptor 2 (TLR2) and Toll-like receptor 4 (TLR4).

20. The method of claim 19, wherein the compound is an antagonist of both TLR2 and TLR4.

21. A method of preventing or reducing edema in a subject in need of treatment thereof, the method comprising administering to the subject, an effective amount of a compound of Formula (I): 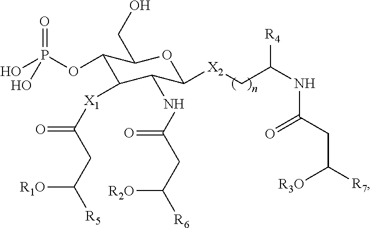
wherein:
n is an integer from 1 to 6;
X₁ is O or S;
X₂ is O or S;
R₁, R₂, and R₃ are independently C₂-C₁₆ acyl, wherein at least one of R₁, R₂, and R₃ is C₂-C₇ acyl;
R₄ is selected from the group consisting of H, hydroxylalkyl, —C(═O)NH₂, and —(CH₂)ₘC(═O)OH, wherein m is an integer from 0 to 2; and
R₅, R₆, and R₇ are independently C₁₀-C₁₂ alkyl, or a pharmaceutically acceptable salt thereof.

22. The method of claim 21, wherein n is 1.

23. The method of claim 21, wherein X₁ and X₂ are each O.

24. The method of claim 21, wherein R₄ is —C(═O)OH.

25. The method of claim 21, wherein R₁, R₂, and R₃ are each C₂-C₇ acyl.

26. The method of claim 21, wherein the compound of Formula (I) is a compound wherein:
n is 1;
X₁ is O;
X₂ is O,
R₁, R₂ and R₃ are each —C(═O)(CH₂)₄CH₃;
R₄ is —C(═O)OH; and
R₅, R₆, and R₇ are each —(CH₂)₁₀CH₃, or a pharmaceutically acceptable salt thereof.

27. The method of claim 21, wherein the edema is related to ischemia-reperfusion.

28. The method of claim 27, wherein the ischemia-reperfusion is related to myocardial infarction or stroke.

29. The method of claim 27, wherein the ischemia-reperfusion is related to cardioplegia during cardiac surgery or to ischemia in skeletal muscle resulting from orthopedic surgery.

30. The method of claim 27, wherein the ischemia-reperfusion is related to organ or tissue transplant.

31. The method of claim 30, wherein the tissue transplant is one of a skin transplant, a muscle transplant, or a soft tissue transplant.

32. The method of claim 31, wherein the tissue transplant is an autologous tissue transplant.

33. The method of claim 27, wherein the compound is administered to the subject prior to a predicted ischemic event, during ischemia, or after an interval of ischemia.

34. The method of claim 21, wherein the compound is an antagonist of one or both of Toll-like receptor 2 (TLR2) and Toll-like receptor 4 (TLR4).

35. The method of claim 34, wherein the compound is an antagonist of both TLR2 and TLR4.

36. A method of preventing or reducing edema related to ischemia-reperfusion in a subject of an organ or tissue transplant, the method comprising:
providing an organ or tissue for transplant;
contacting the organ or tissue with a compound of Formula (I): 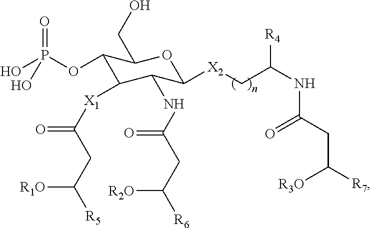
wherein:
n is an integer from 1 to 6;
X₁ is O or S;
X₂ is O or S;
R₁, R₂, and R₃ are independently C₂-C₁₆ acyl, wherein at least one of R₁, R₂, and R₃ is C₂-C₇ acyl;
R₄ is selected from the group consisting of H, hydroxylalkyl, —C(═O)NH₂, and —(CH₂)ₘC(═O)OH, wherein m is an integer from 0 to 2; and
R₅, R₆, and R₇ are independently C₁₀-C₁₂ alkyl, or a pharmaceutically acceptable salt thereof, thereby providing a treated organ or tissue; and
transplanting the treated organ or tissue into a subject in need of said transplant, wherein edema related to ischemia-reperfusion in the subject is prevented or reduced in comparison edema related to ischemia-reperfusion in a subject of a transplant performed using an organ or tissue untreated with said compound.

37. The method of claim 36, wherein the organ or tissue is selected from the group consisting of a heart or heart tissue, a liver or liver tissue, a kidney or kidney tissue, a pancreas or pancreatic tissue, small bowel tissue, large bowel tissue, skin tissue, skeletal muscle tissue, soft tissue, a lung or lung tissue, and brain tissue.

38. The method of claim 37, wherein the organ or tissue is a lung or lung tissue.

39. The method of claim 38, wherein the contacting is performed via one of the airway of a lung tissue donor, the pulmonary vein, and the pulmonary artery of an ex vivo perfusion circuit.

40. The method of claim 37, wherein the tissue is skin tissue, skeletal muscle tissue or soft tissue.

41. The method of claim 40, wherein the tissue transplant is an autologous tissue transplant.

42. The method of claim 36, wherein the organ or tissue is from a non-heart-beating organ donor.

43. The method of claim 36, wherein the compound is an antagonist of one or both of Toll-like receptor 2 (TLR2) and Toll-like receptor 4 (TLR4).

44. The method of claim 43, wherein the compound is an antagonist of both TLR2 and TLR4.

45. The method of claim 36, wherein n is 1.

46. The method of claim 36, wherein X₁ and X₂ are each O.

47. The method of claim 36, wherein R₄ is —C(═O)OH.

48. The method of claim 36, wherein R₁, R₂, and R₃ are each C₂-C₇ acyl.

49. The method of claim 36, wherein the compound is an aminoalkyl glucosaminide phosphate of Formula (I) wherein:
n is 1;
X₁ is O;
X₂ is O;
R₁, R₂ and R₃ are each —C(═O)(CH₂)₄CH₃;
R₄ is —C(═O)OH; and
R₅, R₆, and R₇ are each —(CH₂)₁₀CH₃, or
a pharmaceutically acceptable salt thereof.

50. A preservation solution for treating an ex vivo organ or organ tissue comprising a compound of Formula (I): 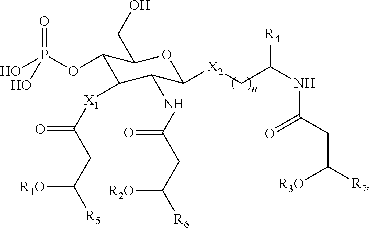
wherein:
n is an integer from 1 to 6;
X₁ is O or S;
X₂ is O or S;
R₁, R₂, and R₃ are independently C₂-C₁₆ acyl, wherein at least one of R₁, R₂, and R₃ is C₂-C₇ acyl;
R₄ is selected from the group consisting of H, hydroxylalkyl, —C(═O)NH₂, and —(CH₂)ₘC(═O)OH, wherein m is an integer from 0 to 2; and
R₅, R₆, and R₇ are independently C₁₀-C₁₂ alkyl, or
a pharmaceutically acceptable salt thereof.

51. The preservation solution of claim 50, wherein n is 1.

52. The preservation solution of claim 50, wherein X₁ and X₂ are each O.

53. The preservation solution of claim 50, wherein R₄ is —C(═O)OH.

54. The preservation solution of claim 50, wherein R₁, R₂, and R₃ are each C₂-C₇ acyl.

55. The preservation solution of claim 50, wherein the compound of Formula (I) is the compound wherein:
n is 1;
X₁ is O;
X₂ is O;
R₁, R₂ and R₃ are each —C(═O)(CH₂)₄CH₃;
R₄ is —C(═O)OH; and
R₅, R₆, and R₇ are each —(CH₂)₁₀CH₃, or
a pharmaceutically acceptable salt thereof.

56. The preservation solution of claim 50, wherein the compound is an antagonist of one or both of Toll-like receptor 2 (TLR2) and Toll-like receptor 4 (TLR4).

57. The preservation solution of claim 56, wherein the compound is an antagonist of both TLR2 and TLR4.

58. The preservation solution of claim 50, wherein the ex vivo organ or tissue is a lung or a portion thereof.
